SEQUENCE LISTING (42 sequences)

SEQ ID NO: 1 - protein, 261 aa, Artificial Sequence
  hsCD40L
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - protein, 215 aa, Artificial Sequence
  hsCD40L (extracellular region)
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 149 aa, Artificial Sequence
  hsCD40L M113 to L261 (portion of soluble)
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 4 - protein, 149 aa, Artificial Sequence
  hsCD140L  Y170A
MQKGDQNPQIAAHVISEASSKTTSVLQWAEKGYYTMSNNLVTLENGKQLTVKRQGLYAIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 149 aa, Artificial Sequence
  hsCD40L G227A
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGAVFELQPGASVFVNVTDPSQVSHGTGFTSFGLLKL

SEQ ID NO: 6 - protein, 149 aa, Artificial Sequence
  hsCD40L Y170A/G227A
MQKGDQNPQIAAHVISEASSKTTSVLQWAEKGYYTMSNNLVTLENGKQLTVKRQGLYAIYAQVTFCSNREASSQAPFIASLCLKSPGRFERILLRAANTHSSAKPCGQQSIHLGAVFELQPGASVFVNVTDPSQVSHGTGFTSFGLLKL

SEQ ID NO: 7 - protein, 260 aa, Artificial Sequence
  mCD40L
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 8 - protein, 210 aa, Artificial Sequence
  smCD40L (from D51extracellular region)
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 9 - protein, 149 aa, Artificial Sequence
  smCD40L (portion)
MQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 10 - protein, 210 aa, Artificial Sequence
  smCD40L Y169
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYAVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 11 - protein, 210 aa, Artificial Sequence
  smCD40L G226
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGAVFELQAGASVFVNVTEASQVIHRVGFSSFGLLKL

SEQ ID NO: 12 - protein, 210 aa, Artificial Sequence
  smCD40L Y169/G226
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYAVYTQVTFCSNREPSSQRPFIVGLWLKPSSGSERILLKAANTHSSSQLCEQQSVHLGAVFELQAGASVFVNVTEASQVIHRVGFSSFGLLKL

SEQ ID NO: 13 - DNA, 25 bp, Artificial Sequence
  5sCD40L-BglII
cgggagatctatgcaaaaaggtgat

SEQ ID NO: 14 - DNA, 22 bp, Artificial Sequence
  3sCD40L-XhoI
tagactcgagtttgagtaagcc

SEQ ID NO: 15 - DNA, 23 bp, Artificial Sequence
  5smCD40L-BglII
cgggagatctgataaggtcgaag

SEQ ID NO: 16 - DNA, 24 bp, Artificial Sequence
  CD40L-Y170A-C
ggcatagatagcatagagtccttg

SEQ ID NO: 17 - DNA, 25 bp, Artificial Sequence
  CD40L-Y170A-B
caacaatccattgcattgggaggag

SEQ ID NO: 18 - DNA, 25 bp, Artificial Sequence
  CD40L-H224A-C
ctcctcccaatgcaatggattgttg

SEQ ID NO: 19 - DNA, 23 bp, Artificial Sequence
  CD40L-H224A-B
cattcacttggcaggagtatttg

SEQ ID NO: 20 - DNA, 23 bp, Artificial Sequence
  CD40L-G226A-C
caaatactcctgccaagtgaatg

SEQ ID NO: 21 - DNA, 24 bp, Artificial Sequence
  CD40L-G226A-B
cacttgggagcagtatttgaattg

SEQ ID NO: 22 - DNA, 24 bp, Artificial Sequence
  CD40L-G227A-C
caattcaaatactgctcccaagtg

SEQ ID NO: 23 - DNA, 22 bp, Artificial Sequence
  CD40L-G227A-B
ggactctatgctgtctacactc

SEQ ID NO: 24 - DNA, 22 bp, Artificial Sequence
  CD40L-Y169A-C
gagtgtagacagcatagagtcc

SEQ ID NO: 25 - DNA, 20 bp, Artificial Sequence
  CD40L-Y169A-B
cagtctgttgcattgggcgg

SEQ ID NO: 26 - DNA, 20 bp, Artificial Sequence
  CD40L-H223A-C
ccgcccaagtcaacagactg

SEQ ID NO: 27 - DNA, 22 bp, Artificial Sequence
  CD40L-H223A-B
gtctgttcacttggccggagtg

SEQ ID NO: 28 - DNA, 22 bp, Artificial Sequence
  CD40L-G225A-C
cactccggccaagtgaacagac

SEQ ID NO: 29 - DNA, 22 bp, Artificial Sequence
  CD40L-G225A-B
gttcacttgggcgcagtgtttg

SEQ ID NO: 30 - DNA, 22 bp, Artificial Sequence
  CD40L-G226A-C
caaacactgcgcccaagtgaac

SEQ ID NO: 31 - DNA, 20 bp, Artificial Sequence
  CD40L-G226A-B
gccaaggagtgctaaagaac

SEQ ID NO: 32 - DNA, 24 bp, Artificial Sequence
  IL-8F
cactggcatcttcactgattcttg

SEQ ID NO: 33 - DNA, 20 bp, Artificial Sequence
  IL-8R
aatctggcaccacaccttct

SEQ ID NO: 34 - DNA, 20 bp, Artificial Sequence
  B-actin F
taatgtcacgcacgatttcc

SEQ ID NO: 35 - DNA, 18 bp, Artificial Sequence
  B-actin R
tctccacaagcgccttcg

SEQ ID NO: 36 - DNA, 18 bp, Artificial Sequence
  IL-6 Sens
ctgagggctgagatgccg

SEQ ID NO: 37 - protein, 149 aa, Artificial Sequence
  IL-6 anti-sense
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIALGGVFELQPGASVFVNVTDPSQVSHGTGFTSFGLLKL

SEQ ID NO: 38 - protein, 149 aa, Artificial Sequence
  Soluble Human H224
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLAGVFELQPGASVFVNVTDPSQVSHGTGFTSFGLLKL

SEQ ID NO: 39 - protein, 149 aa, Artificial Sequence
  Soluble Human G226
MQKGDQNPQIAAHVISEASSKTTSVLQWAEKGYYTMSNNLVTLENGKQLTVKRQGLYAIYAQVTFCSNREASSQAPFIASLCLKSPGRFERILLRAANTHSSAKPCGQQSIALGGVFELQPGASVFVNVTDPSQVSHGTGFTSFGLLKL

SEQ ID NO: 40 - protein, 149 aa, Artificial Sequence
  Soluble Human Y170/H224
MQKGDQNPQIAAHVISEASSKTTSVLQWAEKGYYTMSNNLVTLENGKQLTVKRQGLYAIYAQVTFCSNREASSQAPFIASLCLKSPGRFERILLRAANTHSSAKPCGQQSIHLAGVFELQPGASVFVNVTDPSQVSHGTGFTSFGLLKL

SEQ ID NO: 41 - protein, 210 aa, Artificial Sequence
  Soluble Human Y170/G226
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVALGGVFELQAGASVFVNVTEASQVIHRVGFSSFGLLKL

SEQ ID NO: 42 - protein, 210 aa, Artificial Sequence
  Soluble Murine H223
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLAGVFELQAGASVFVNVTEASQVIHRVGFSSFGLLKL

(19) United States
(12) Patent Application Publication
(10) Pub. No.: US 2010/0150931 A1
(43) Pub. Date: Jun. 17, 2010

(54) NOVEL RECEPTOR FOR CD40L AND USES THEREOF

(75) Inventors: Walid Mourad, Laval (CA); Claire Léveillé, Ste-Foy (CA); Youssef El Fakhry, Saint-Laurent (CA); Djibril Diallo, Saint-Hubert (CA); Haydar Alturaihi, Brossard (CA)

Correspondence Address: BCF LLP, 1100 RENE'-LE'VESQUE BLVD. WEST, 25TH FLOOR, MONTREAL, QC, H3B-5C9, CA

(73) Assignee: CENTRE HOSPITALIER DE L'UNIVERSITE DE MONTREAL, Montreal, QC (CA)

(21) Appl. No.: 12/312,708

(22) PCT Filed: Nov. 22, 2007
(86) PCT No.: PCT/CA2007/002124

§ 371 Date: Mar. 2, 2010

Related U.S. Application Data
(60) Provisional application No. 60/866,986, filed on Nov. 22, 2006.

(51) Int. Cl. A61K 39/395; A61K 38/02; C07K 14/705; A61K 38/17; G01N 33/566; G01N 33/573

(52) U.S. Cl. 424/141.1; 514/2; 424/130.1; 530/395; 424/158.1; 514/12; 436/501; 435/7.4

(57) ABSTRACT

The present invention relates to a novel CD40L receptor and its related activities. Methods, uses, reagents and kits for the modulation of CD40L activities related to its interaction with the novel receptor are disclosed. Also disclosed are therapeutic uses of reagents in treating CD40L-related disorders.

### FIELD OF INVENTION

The present invention relates to a novel receptor for CD40L and to CD40L-related activity. More specifically, the present invention relates to methods, uses, reagents and kits for the modulation of CD40L-related activity. The present invention also relates to new agents for treating CD40L-related disorders.

### BACKGROUND OF THE INVENTION

Human CD40 ligand (CD40L) SEQ ID NO.1, also known as CD154, gp39 and TRAP, is a 33 kDa type II transmembrane protein composed of 261 amino acids (a.a.) with a 215 a.a. extracellular domain SEQ ID NO.2, a 24 a.a. transmembrane domain, and a 22 a.a. intracellular tail (1). Human CD40L shares high homology with the murine form of CD40L. It belongs to the tumor necrosis factor (TNF) superfamily and was initially described as a molecule transiently expressed mainly on activated CD4-positive T cells. Later, it was shown that CD40L is also expressed on immune cells such as mast cells, basophils, eosinophils, natural killer cells, activated B cells, as well as activated platelets. Expression of CD40L differs according to cell type and type of stimuli. The expression of CD40L is inducible, and its expression on T cells is triggered primarily by TCR signaling and is regulated by CD28-dependent and independent pathways. CD40L is stored in platelets and a subpopulation of T cells and rapidly translocates to the cell membrane following T cell and platelet activation. Like other members of the TNF superfamily, the extracellular domain of CD40L forms a homotrimer. Soluble trimeric CD40L (sCD40L), which is biologically active, is released from activated T cells by proteolytic cleavage, but the physiological role of sCD40L in vivo remains unclear.

The first characterized receptor for CD40L is CD40. The CD40 molecule is a 45-50 kDa phosphorylated type I integral membrane glycoprotein that belongs to the tumor necrosis factor receptor (TNFR) superfamily (2). CD40 was initially considered a pan B cell antigen but was subsequently shown to be expressed on a number of cell types, both constitutively and following activation (3). The engagement of CD40 triggers several events that are not restricted to cells of the immune system but also involve other cell types such as epithelial cells and fibroblasts (2). Cross-linking CD40 triggers the secretion of a number of cytokines (TNFα, LTα, LTβ, IL-6, and IL-10), results in a higher expression of CD23, MHC class II, B7 proteins and BB1, and has a co-mitogenic effect on resting B cells stimulated with anti-IgM or phorbol esters. Simultaneous triggering of BCR and CD40 on resting B cells results in differentiation and the production of IgM, IgG, and IgA, but not IgE unless exogenous cytokines are added. The engagement of CD40 rescues immature B cells from BCR-induced cell death, prevents the apoptotic death of germinal center B cells, and contributes to the development of B cell memory. Several signaling pathways and second messengers have been reported to mediate these cellular events, including PTKs such as lyn, fyn, and syk, phosphatidylinositol-3-kinase (PI3 kinase), PLCγ2, Jak3, p38, and JNK mitogen-activated protein kinase (MAPK) (4).

An indication of the pivotal role of the CD40L/CD40 interaction in T cell-dependent B cell responses in vivo emerged from studies of X-linked Hyper IgM syndrome (HIM), which is characterized by point mutations or deletions in the gene coding for CD40L. Patients suffering from HIM do not produce antibodies to exogenous antigens, but produce large amounts of auto-antibodies and are susceptible to various infections. The key role of CD40L/CD40 interactions in T cell-dependent B cell activation has been confirmed with the development of CD40 and CD40L knockout mice. The possible role of the CD40/CD40L interaction in the development of autoimmune diseases such as collagen-induced arthritis (CIA), experimental allergic encephalomyelitis (EAE), oophoritis, lupus nephritis, and colitis has also been addressed. It has been demonstrated, for example, that treatment with anti-CD40L Abs inhibits the development of these diseases (5; 6). This inhibitory effect may be due to the blocking of priming self-antigen-specific T cells, the inhibition of the effector function of CD40L such as the activation of CD40-positive cells, and/or the blocking of the humoral response.

In addition to its well-characterized receptor, CD40, there is evidence to indicate that human and murine CD40L specifically bind to αllbβ3 integrin (7; 8), a glycoprotein highly and mainly expressed on platelets (9). On unstimulated platelets, αllbβ3, like most integrins, is not constitutively active having low affinity for its ligands fibrinogen and von Willebrand factor (10). However, platelet activation by agents such as thrombin, triggers signals that cause the activation of the integrin resulting in conformational changes that increase the binding affinity for its ligands (11). Ligation of αllbβ3 with sCD40L induces β3 integrin phosphorylation and platelet activation (8).

Several pre-clinical studies have demonstrated the promise of agents capable of modulating the CD40/CD40L interaction for the prevention/treatment of immune-mediated diseases. For example, it is well established that disruption of CD40/CD40L interactions using anti-CD40L monoclonal antibodies (mAbs) in animal models and in clinical trials prevent disease development and possibly reverse established disease such as autoimmune diseases (5; 6). However, some adverse effects have been reported following anti-CD40L-based treatment in non-human primates and humans.

Therefore, there is a need for novel reagents and methods based on CD40L for the prevention/treatment of diseases/conditions associated with CD40L activity.

The present description refers to a number of documents, the content of which is herein incorporated by reference in their entirety.

### SUMMARY OF THE INVENTION

The invention relates to various methods, uses, reagents, and kits based on modulation of the interaction between CD40L and α5β1 integrin.

In one aspect thereof, the present invention relates to an agent that may block the interaction between CD40L and α5β1 integrin.

In another aspect, the present invention relates to a composition that may comprise an agent that may block the interaction between CD40L and α5β1 integrin and a pharmaceutically acceptable carrier.

The present invention further relates to a method for blocking the interaction between CD40L and α5β1 integrin; the method may comprise the step of administering an effective amount of an agent that may block the interaction between CD40L and α5β1 integrin.

In an embodiment of the present invention, the interaction may occur at the cell surface and a method for blocking the cell surface interaction between CD40L and α5β1 integrin may comprise contacting cells (a cell expressing CD40L and/or a cell expressing α5β1 integrin) with an effective amount of an agent that may block the interaction between CD40L and α5β1 integrin.

In a further aspect, the present invention provides a method for inhibiting production of an inflammatory mediator by a cell, the method may comprise blocking the interaction between CD40L and α5β1 integrin.

In another aspect, the present invention relates to the use of an agent that may block the interaction between CD40L to α5β1 integrin and/or for the preparation of a medicament that may block the interaction between CD40L and α5β1 integrin.

In a further aspect, the present invention relates to the use of an agent for treating an inflammatory disease in a subject and/or for the preparation of a medicament for treating an inflammatory disease in a subject.

In yet a further aspect, the present invention relates to a method for identifying a compound capable of blocking the interaction between CD40L and α5β1 integrin; the method may comprise measuring the binding of CD40L to α5β1 integrin in the presence versus the absence of an agent, wherein a lower binding of CD40L to α5β1 integrin in the presence of the agent (in comparison with the absence of the agent) may be indicative that the agent is capable of blocking the interaction between CD40L and α5β1 integrin.

In another aspect, the present invention relates to a method for identifying a compound capable of blocking the interaction between CD40L and α5β1 integrin; the method may comprise measuring a CD40L-mediated α5β1 integrin activity in the presence or absence of the agent, wherein a lower α5β1 integrin activity in the presence of the agent may be indicative that the agent is blocking the interaction between CD40L and α5β1 integrin.

In another aspect, the present invention relates to a method for identifying a compound capable of inhibiting and/or decreasing inflammation; the method may comprise measuring the binding of CD40L to α5β1 integrin in the presence versus the absence of the agent, wherein a lower binding of CD40L to α5β1 integrin in the presence of the agent may be indicative that the agent is capable of inhibiting or decreasing inflammation.

In yet another aspect, the present invention provides a method of identifying a compound capable of inhibiting or decreasing inflammation; the method may comprise measuring a CD40L-mediated α5β1 integrin activity in the presence versus the absence of the agent, wherein a lower α5β1 integrin activity in the presence of the agent may be indicative that the agent is capable of inhibiting or decreasing inflammation.

In a further aspect, the present invention provides a method of treating an inflammatory disease or condition in a subject; the method may comprise blocking the interaction between CD40L and α5β1 integrin in the subject.

In another aspect, the present invention relates to a method for treating thrombosis in a subject (in need thereof), the method may comprise administering an agent that may block the interaction between CD40L and α5β1 integrin as described herein.

In another aspect, the present invention relates to a method for treating asthma in a subject (in need thereof), the method may comprise administering an agent that may block the interaction between CD40L and α5β1 integrin as described herein.

In another aspect, the present invention relates to a method for treating bronchial hyperresponsiveness in a subject (in need thereof), the method may comprise administering an agent that may block the interaction between CD40L and α5β1 integrin as described herein.

In a further aspect, the present invention related to a use of an agent capable of blocking the interaction between CD40L and α5β1 integrin for treating an inflammatory disease or condition in a subject.

In a further aspect, the present invention relates to a use of an agent capable of blocking the interaction between CD40L and α5β1 integrin for the preparation of a medicament for treating an inflammatory disease or condition in a subject.

In a further aspect, the present invention relates to a composition for treating an inflammatory disease or condition in a subject comprising an agent capable of blocking the interaction between CD40L and α5β1 integrin and a pharmaceutically acceptable carrier.

In a further aspect, the present invention relates to a method of identifying a compound capable of controlling immune activation and inflammation without inducing a blood-related disorder, the method comprising:
(a) measuring a first binding or activity of CD40L to CD40 in the presence versus the absence of the agent;
(b) measuring a second binding or activity of CD40L to α5β1 integrin in the presence versus the absence of the agent,
(c) measuring platelet aggregation or activation in the presence of the agent;
   wherein a lower first and second binding or activity and a lower platelet activation or aggregation in the presence of the agent may be indicative that the agent is capable of controlling immune activation and inflammation without inducing a blood-related disorder.

In a further aspect, the present invention relates to a package comprising:
(a) an agent capable of blocking the interaction between CD40L and α5β1 integrin; and
(b) instructions for its use.

In an embodiment, the use of a package may be for the treatment and/or prevention (reduction, blockade, inhibition) of inflammatory-related diseases or condition in the subject.

In another aspect thereof, the present invention relates to a monomeric soluble form of CD40L such as hCD40L mutated in its Y170 residue SEQ ID NO.4, hCD40L mutated in its G227 residue SEQ ID NO.5, hCD40L mutated in both its Y170 and G227 residue SEQ ID NO.6, mCD40L mutated in its Y169 residue SEQ ID NO.10, mCD40L mutated in its G226 residue SEQ ID NO.11, mCD40L mutated in both its Y169 and G226 SEQ ID NO. 12 residue and/or portion thereof.

The present invention also relates to the use of monomeric soluble form(s) of CD40L for treatment of CD40L-related disorders as well as a method for treating CD40L-related disorders; the method may comprise administering monomeric soluble form of CD40L. Monomeric soluble form(s) of CD40L of the present invention may also be used for detecting a CD40L receptor by methods well within the province of a person skilled in the art.

Other objects, advantages and features of the present invention will become more apparent upon reading the following non-restrictive description of specific embodiments thereof, given by way of example only with reference to the accompanying drawings.

### BRIEF DESCRIPTION OF THE DRAWINGS

In drawings which illustrates non-limitative exemplary embodiments of the present invention,

FIG. 1 shows that rsCD40L binds to U937 cells in a CD40-independent manner. (A) Flow cytometric analysis of CD40 surface expression. Cells were stained with anti-CD40 mAb G28.5 or isotype control anti-TSST1 mAb 2H8 followed by FITC-labeled goat anti-mouse IgG antibody. BJAB cells were used as a positive control. (B) rsCD40L-A bound to CD40-negative U937 cells. Cells were incubated for 1 h at 37° C. with rsCD40L-A in the presence (sCD40L) or absence of unlabelled rsCD40L, washed, and analyzed by FACS. Avidin-A was used as a control. (C) Neutralizing anti-CD40L mAb 5C8 prevented the binding of rsCD40L-A to BJAB and U937 cells. rsCD40L-A and avidin-A were incubated with mAb 5C8 or isotype control for 30 min at 37° C. prior to the addition of BJAB and U937 cells. Similar results were obtained with avidin-A+isotype control and avidin-A+5C8 (avidin-A). (D) Blocking anti-CD40 mAb 82102 did not prevent the binding of rsCD40L-A to U937 cells. Cells were incubated with a saturating amount of mAb 82102 prior to incubation with rsCD40L-A or avidin-A. This figure is representative of three independent experiments;

FIG. 2 shows that rsCD40L-A binding to α5β1 -positive U937 cells but not to α5β1-negative BJAB cells is prevented by soluble α5β1 (sα5β1) and anti-α5 mAb P1D6. (A) Flow cytometric analysis of α5β1 integrin surface expression. Cells were incubated with anti-α5β1 mAb HA5 or isotype control mAb followed by FITC-labeled goat anti-mouse IgG Ab and analyzed by FACS. (B) Soluble α5β1 prevented the binding of rsCD40L-A to U937 cells but not to BJAB cells. rsCD40L-A was pre-incubated with sα5β1 or not for 1 h at 37° C. prior to the addition of cells. Similar results were obtained with avidin-A and avidin-A+sα5β1 (avidin-A). (C) Pre-incubation of U937 cells with anti-α5β1 mAb P1 D6 significantly prevented the binding of rsCD40L-A. Cells were incubated with mAb P1 D6 or an isotype control (IgG3) for 30 min at 37° C. Avidin-A or rsCD40L-A was then added and the incubation continued for 1 h at 37° C. This figure is representative of three independent experiments;

FIG. 3 shows that immobilized purified α5β1 binds rsCD40L; (A) rsCD40L bound to recombinant soluble CD40-Fc in a dose-dependent manner in a solid phase binding assay. The wells of microtiter plates were coated with 4 μg/ml recombinant soluble CD40-Fc (square), or with BSA alone (dot) as a control. rsCD40L binding was detected with anti-CD40L-biotin antibody and streptavidin-HRP and was revealed with TMB. (B) α5β1 is a receptor for rsCD40L. The wells of microtiter plates were coated with purified sα5β1 (4 μg/ml) (square) or BSA (2%) (dot) and rsCD40L was added at the indicated concentrations. Bound rsCD40L was detected as above. The results presented in this figure (mean value of duplicate wells) are representative of three independent experiments. Standard deviation (SD) was less than 5% of the mean;

FIG. 4 shows that Mn²⁺ and DTT treatments induce the adherence of U937 cells but not BJAB cells to fibronectin. (A) Cells were treated with Mn²⁺ (1 mM) or DTT (10 mM) or not for 30 min at room temperature. Washed cells were then added to fibronectin-coated wells and allowed to adhere for 1 h at 37° C. After removing unbound cells, adherence was assessed using a Zeiss microscope. (B) Colorimetric evaluation of cells adhering to fibronectin. Treated or untreated cells were tested for their adherence to immobilized fibronectin as described above. After removing unbound cells, a colorimetric assay was used as described in Example 1 (Material and methods) to evaluate the adherence of U937 and BJAB cells to fibronectin. These results are representative of four independent experiments;

FIG. 5 shows that treating U937 cells with Me²⁺ or DTT exposes the B44 β1 epitope but decreased the binding of rsCD40L to α5β1. (A) Effects of Mn²⁺ and DTT treatments of U937 and BJAB cells on B44 β1 epitope expression. Cells were treated with Mn²⁺ and DTT as indicated above, and the expression of the B44 β1 epitope was assessed by flow cytometry. (B) Treatment of U937 cells with Mn²⁺ or DTT prevented the binding of rsCD40L-A. Cells were treated with Mn²⁺ or DTT as described above, and the binding of rsCD40L-A was assessed as described in FIG. 1. These results are representative of four independent experiments;

FIG. 6 shows that stimulating U937 cells with rsCD40L leads to the recruitment of α5β1 into the Triton™-X-100 insoluble fraction. Cells were stimulated for 30 min at 37° C. with rsCD40L and then lysed in 1% Triton™ X-100 buffer. Cell lysates were centrifuged at 16,000×g for 20 min at 4° C. Soluble (10⁶ cell equivalents) and insoluble (2×10⁶ cell equivalents) fractions were separated by SDS-PAGE under non-reducing conditions and analyzed by immunoblotting using anti-human α5 (A) and anti-human CD40 (B). Med is an acronym for medium/media. This figure is representative of three independent experiments;

FIG. 7 shows that rsCD40L induces the phosphorylation of pERK1/2 in U937 cells and this response was prevented by sα5β1. (A) Cells (5×10⁵) were stimulated at 37° C. with rsCD40L in serum-free medium and then lysed in 2× SDS sample buffer at the indicated time points. (B) rsCD40L was incubated with soluble α5β1 (+sα5β1) or not for 1 h at 37° C. before adding the cells (5×10⁵) in serum-free medium. After 5 min at 37° C. the cells were centrifuged and lysed in 1× SDS sample buffer. Immunoblotting was performed with antibodies (Abs) specific for phosphorylated ERK1/2 and reprobed with Abs specific for total ERK1/2. This figure is representative of three independent experiments;

FIG. 8 shows that sCD40L induces the expression of a chemokine, IL-8, in CD40-negative α5β1-positive cells. U937 cells (2×10⁶/100 μl) were stimulated with 100 ng of rsCD40L for 15, 60, 120 and 240 min at 37° C. Cells were harvested, total RNA extracted and IL-8 gene expression was analyzed by RT-PCR with IL-8 specific primers. (A) PCR products were electrophoresed in 1% agarose gel, stained with ethidium bromide and visualized with a Molecular Imager Gel Doc System. (B) The fluorescence intensity of PCR products of each sample was used to evaluate the induced IL-8 gene expression. The results show that a significant expression of IL-8 mRNA was rapidly induced in U937 cells by sCD40L with a maximal response (2.5 fold increase in gene expression) at two hours;

FIG. 9 shows that sCD40L induces the expression of metalloproteinases in CD40-negative α5β1-positive cells. U937 cells were incubated with rsCD40L (1 μg/ml), PMA (100 ng/ml), medium alone (medium) or co-cultured with LTK, LTK-CD40L (1:5 ratio) or LTK-CD40L*(1:1 ratio) for 48 h at 37° C. in RPMI+10% FBS. Cell culture supernatants were assayed for MMP activity by zymography (A) or proMMP-1 secretion by ELISA (B);

FIG. 10 shows the Commassie Blue staining (A) and Western blot analysis (B) of purified CD40L monomeric mutants. For Western blot, 100 ng of protein was loaded on 10% SDS-PAGE gel, transferred to membrane, and blotted with rabbit anti-CD40L Ab, followed by anti-rabbit HRP conjugated Ab;

FIG. 11 shows the functional analysis of CD40L monomeric mutants. BJAB or U937 cells were stimulated for 5 min with wt or mutants CD40L at 300 ng and activity was monitored by detection of p38 phosphorylation. CD40L mutants failed to induce p38 phosphorylation in BJAB (A) and U937 cells (B);

FIG. 12 shows that binding of CD40L-Alexa to U937 cells and BJAB cells is blocked by mutant monomeric CD40L (Y170 and G227). U937 (A) or BJAB (B) cells were incubated for 1 h with sCD40L-Alexa (20 ng/sample) in the absence or in the presence of CD40L-Y170 SEQ ID NO.4 (200 ng), or CD40L-G227 SEQ ID NO. 5. Washed cells were then analyzed by flow cytometry;

FIG. 13 shows thrombosis in mice in the presence or absence of anti-CD40 or anti-CD40L mAbs. Thrombosis formation was monitored in carotid arteries of C57Bl/6 mice, and induced by FeCl₃. The graph represents the amplitude (%) of blood flow plotted against time for the different treatments groups;

FIG. 14 shows thrombosis in mice in the presence or absence of soluble monomeric murine CD40L SEQ ID NO.7. Thrombosis formation was monitored in carotid arteries of C57B16 mice, and induced by FeCl₃. Mice were treated, prior to the injury with saline (control), soluble monomeric murine CD40 ligand (Y169 SEQ ID NO. 10, G226 SEQ ID NO.11 or Y169/G226 SEQ ID NO. 12). The graph represents the amplitude (%) of blood flow plotted against time for the different treatments groups;

FIG. 15 show result of a bronchial hyper responsiveness experiments. Mice were sensitized and challenged with OVA, and treated with 5 intranasal instillations of 20 μg anti-CD40L (MR1 clone) or anti-CD40 (FGK clone) antibody. Pulmonary resistance was measured by flexiVent, after increasing concentrations of aerosolized methacholine. Data are compared to typical levels of hyper responsiveness seen in sensitized and challenged animals from previous experiments;

FIG. 16 shows results of IL-6 expression from co-cultures of bronchial fibroblasts and T cells in the presence or the absence of CD40L antibody;

FIG. 17 shows a sequence alignment between human and murine CD40L full length protein SEQ ID NOs. 1 and 7 (pubmed acces cod NP_{—}000065 and NP_{—}035746); residues hY170/mY169, hH224/mH223, hG226/mG225 and hG227/mG226 are in depicted bold character.

### DETAILED DESCRIPTION OF THE INVENTION

In order to provide a clear and consistent understanding of the terms used in the present disclosure, a number of definitions are provided below. Moreover, unless defined otherwise, all technical and scientific terms as used herein have the same meaning as commonly understood to one of ordinary skill in the art to which this invention pertains.

In the studies described herein, Applicants have demonstrated that the α5β1 integrin (also known as very late antigen 5 (VLA-5), CD49e/CD29) is a functional receptor for CD40L and that CD40L induces the production of inflammatory mediators by α5β1-expressing cells.

α5β1 integrin is a widely distributed cell surface receptor that binds to the extracellular matrix through fibronectin and, in doing so, provides cells with adhesive properties and a transmembrane link between the extracellular environment and the intracellular cytoskeleton (13). Recent studies indicate a role of α5β1 in the production of pro-inflammatory mediators and in the induction or progression of inflammation-related diseases such as arthritis (14, 15).

In one aspect thereof, the present invention relates to an agent that may block the interaction between CD40L and α5β1 integrin.

In an embodiment of the present invention, an “agent” that may block the interaction between CD40L and α5β1 integrin may be a protein. For example, such protein may be an (isolated) antibody, or antigen-binding fragment (portion) thereof, that may specifically bind to CD40L and/or α5β1. The antibody may be, for example, a monoclonal antibody and/or a polyclonal antibody. Monoclonal antibodies (MAbs) may be made by one of several procedures available to one of skill in the art, for example, by fusing antibody producing cells with immortalized cells and thereby making a hybridoma. The general methodology for fusion of antibody producing B cells to an immortal cell line is well within the province of one skilled in the art. Another example is the generation of MAbs from mRNA extracted from bone marrow and spleen cells of immunized animals using combinatorial antibody library technology. One drawback of MAbs derived from animals or from derived cell lines is that although they may be administered to a patient for diagnostic or therapeutic purposes, they are often recognized as foreign antigens by the immune system and are unsuitable for continued use. Antibodies that are not recognized as foreign antigens by the human immune system have greater potential for both diagnosis and treatment. Methods for generating human and humanized antibodies are now well known in the art.

Polyclonal antibodies may be obtained by immunizing a selected animal with a protein or polypeptide (for example without limitation CD40L and α5β1 integrin). Serum from the animal may be collected and treated according to known procedures. Polyclonal antibodies to the protein or polypeptide of interest may then be purified by affinity chromatography. Techniques for producing polyclonal antisera are well known in the art.

Antibodies may originate for example, from a mouse, rat or any other mammal. The antibody may also be a human antibody which may be obtained, for example, from a transgenic non-human mammal capable of expressing human immunoglobulin genes. The antibody may also be a humanized antibody which may comprise, for example, one or more complementarity determining regions of non-human origin. It may also comprise a surface residue of a human antibody and/or framework regions of a human antibody. The antibody may also be a chimeric antibody which may comprise, for example, variable domains of a non-human antibody and constant domains of a human antibody. Suitable antibodies may also include, for example, an antigen-binding fragment, a Fab fragment; a F(ab')2 fragment, and Fv fragment; or a single-chain antibody comprising an antigen-binding fragment (e.g., a single chain Fv). An antibody encompassed in the present invention may be an antibody binding specifically to α5β1 integrin. In an embodiment, an antibody encompassed in the present invention may be an antibody binding specifically to CD40L.

Anti-CD40L agents (e.g. antibodies) may be experimentally tested and validated using in vivo and in vitro assays. Suitable assays include, but are not limited to, activity assays and binding assays. For example, assays for testing CD40L activity for CD40 includes B-cell proliferation assays (20), the NF-KB pathway activation assays (21), c-Jun (22) transcription factor activation assays, or B cell surface receptor activation-induced apoptosis rescue assay (23) for monitoring signaling through CD40 are screens that may be utilized in identifying anti-CD40L antibodies and CD40L variants that have agonists or antagonists properties for CD40.

According to the present invention, a (protein) agent may also be a “soluble protein”. Soluble proteins (purified) of the invention may be obtained from any techniques well known in the art. For example, a soluble protein may be obtained by transfecting a recombinant DNA molecule expressing solely the extracellular region of a molecule and/or portion thereof followed by purification. In another example, a protein and/or a portion of a protein (for example an extracellular region exempt of its transmembrane and cytoplasmic domains) may be fused to a constant domain (Fc portion) of an immunoglobulin. A (purified) soluble protein of the present invention may be soluble CD40L and/or soluble α5β1 integrin and/or portion thereof. By “portion” (of soluble protein for example) it is meant a portion that exhibits similar (biological) activity yet is smaller in size. An agent of the present invention may be soluble α5β1 integrin. An agent of the present invention may be portions of soluble α5β1 integrin. An agent of the present invention may be soluble CD40L. An agent of the present invention may be portions of soluble CD40L. Human CD40L SEQ ID NO.1 is a 261 amino acid type II protein. Its extracellular domain is encoded starting at residue 47 SEQ ID NO.2. A soluble trimeric form of hCD40L exists physiologically (81). A (purified) soluble human CD40L of the invention may have a sequence that may consist in about residue 47 to residue 261. In an embodiment, a soluble human CD40L portion may encompass a sequence from after residue 47 to residue 261 (or less). The present invention relates to and explicitly incorporates herein each and every specific member and combination of sub-ranges therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. For example, the (recombinant; purified) soluble human CD40L portion may encompass the sequence starting at residue 113 (M) to 261 (L) SEQ ID NO.3. Murine CD40L SEQ ID NO.7 is a 260 amino acid type II protein. A soluble trimeric form of mCD40L exists physiologically. A (purified) soluble murine CD40L of the invention may encompass residues 51 (D) to 260 (L) SEQ ID NO.8 or portion thereof SEQ ID NO.9.

In an embodiment, soluble CD40L (human, murine) may be monomeric, dimeric and/or trimeric CD40L and portion thereof. In an embodiment of the present invention, soluble CD40L is monomeric and may be used as an agent to block the interaction between CD40L and α5β1 integrin. Monomeric soluble CD40L may serve to block the interaction with CD40L and α5β1 integrin (and other receptors) with the advantage of acting as an antagonist. Monomeric CD40L of the invention may be (soluble) CD40L in which residues located at position Y170, H224, G226 and/or G227 in human CD40L sequence are mutated to another (conservative and/or non-conservative) residue. An embodiment of a non-conservative residue replacement for any of Y170, H224, G226 and/or G227 is alanine. It is also known in the art that alanine is equivalent to valine, leucine and/or isoleucine (see Table 1). In an embodiment, (purified; soluble) monomeric CD40L is mutated in the Y170 position SEQ ID NO.4. In an embodiment, (soluble) monomeric CD40L is mutated in the G227 position SEQ ID NO.5. In an embodiment, (soluble) monomeric CD40L is mutated in both its Y170 and G227 position SEQ ID NO.6. As described above, mutated means that the original residue may be replaced by another residue. In an embodiment, a replacement residue may be an alanine residue or other replacement residues. For example, conservative substitution/replacement yielding soluble monomeric CD40L with substantially similar activity is within the scope of this invention. Naturally occurring residues are divided into groups based on common side chain properties:
(1) hydrophobic: norleucine, methionine (Met), Alanine (Ala), Valine (Val), Leucine (Leu), Isoleucine (Ile)
(2) neutral hydrophilic: Cysteine (Cys), Serine (Ser), Threonine (Thr)
(3) acidic: Aspartic acid (Asp), Glutamic acid (Glu)
(4) basic: Asparagine (Asn), Glutamine (Gin), Histidine (His), Lysine (Lys), Arginine (Arg)
(5) residues that influence chain orientation: Glycine (Gly), Proline (Pro); and aromatic: Tryptophan (Trp), Tyrosine (Tyr), Phenylalanine (Phe)

A conservative substitution will entail exchanging a member of one group with another member of the same group. Non-conservative substitutions will entail exchanging a member of one of these groups for another.

TABLE 1
AMINO ACID SUBSTITUTION

| ORIGINAL | EXEMPLARY | CONSERVATIVE |
|---|---|---|
| RESIDUE | SUBSTITUTION | SUBSTITUTION |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

Other substitutions may be generated by substitutional mutagenesis and retain the activity of (soluble) monomeric CD40L. These analogs have at least one amino acid residue in the protein molecule removed and a different residue inserted in its place. Such mutagenesis is well within the province of a person skilled in the art and is within the scope of the present invention.

In as aspect thereof, the present invention relates to a (purified) monomeric soluble form of CD40L such as hCD40L mutated in its Y170 residue SEQ ID NO.4, hCD40L mutated in its G227 residue SEQ ID NO.5, hCD40L mutated in both its Y170 and G227 residue SEQ ID NO.6, mCD40L mutated in its Y169 residue SEQ ID NO. 10, mCD40L mutated in its G226 residue SEQ ID NO.11, mCD40L mutated in both its Y169 and G226 residue SEQ ID NO.12 and/or portion thereof. In an embodiment, mutation is replacement of the residue to an alanine residue however; other replacement residues yielding (soluble) monomeric CD40L with substantially similar activity are within the scope of this invention.

The present invention also relates to the use of (purified) monomeric soluble form(s) of CD40L for blocking the interaction of CD40L with its receptors (CD40, αllbβ3, α5β1) and/or for treatment of CD40L-related disorders as described above as well as a method for treating CD40L-related disorders; the method may comprise administering monomeric soluble form of CD40L. Monomeric soluble form(s) of CD40L of the present invention may be labeled with a reporter molecule by methods well within the province of a person skilled in the art. Labeled monomeric soluble forms of CD40L may be used for detecting a CD40L receptor.

As used herein, the term “block” or “inhibit” refers to a decrease in one or more given measurable activity by at least 10% relative to a reference and/or control. Where inhibition is desired, such inhibition is preferably at least 20%, 30%, 40%, 50%, 60%, 70%, 80%, 90% or more, up to and including 100%, i.e., complete inhibition or absence of the given activity. As used herein, the term “substantially inhibits/blocks” refers to a decrease in a given measurable activity by at least 50% relative to a reference. For example, “substantially inhibits” refers to a decrease in a given measurable activity of at least 50%, 55%, 60%, 65%, 70%, 75%, 80%, 85%, 90%, 95% and up to and including 100% relative to a reference. As used herein, “blocks/prevents/inhibits/impairs/lowers the interaction”, with reference to the binding of CD40L to a receptor refers to a decrease in binding by at least 10% relative to a reference. An agent may block the binding of CD40L to α5β1 integrin expressing cells. “Inhibits the interaction” and/or “block the binding” preferably refers to a decrease in binding of at least 20%, 30%, 40%, 50%, 60%, 70%, 80%, 90% or more, up to and including 100%.

In another aspect, the present invention relates to a composition including an agent that blocks the interaction between CD40L and α5β1 integrin and a pharmaceutically acceptable carrier.

A “composition” of the invention including an agent may be manufactured in a conventional manner. In particular, it is formulated with a pharmaceutically acceptable diluent or carrier, e.g., water or a saline solution such as phosphate buffer saline. In general, a diluent or carrier is selected on the basis of the mode and route of administration, as well as standard pharmaceutical practice. Therapeutic compositions typically must be sterile and stable under the conditions of manufacture and storage. The composition can be formulated as a solution, microemulsion, liposome, or other ordered structure suitable to high drug concentration. The proper fluidity can be maintained, for example, by the use of a coating such as lecithin, by the maintenance of the required particle size in the case of dispersion and by the use of surfactants. In many cases, it may be preferable to include isotonic agents, for example, sugars, polyalcohols such as mannitol, sorbitol, or sodium chloride in the composition. Prolonged absorption of compositions may be brought about by including in the composition an agent which delays absorption, for example, monostearate salts and gelatin. Moreover, an agent of the invention may be administered in a time release formulation, for example in a composition which includes a slow release polymer. The active agents may be prepared with carriers that will protect the agent against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the relevant art. The present invention relates to compositions that may comprise an agent capable of modulating CD40L activity and a pharmacologically acceptable carrier. In one embodiment, such compositions include an agent that may block the interaction between CD40L and α5β1 integrin to treat a CD40L-related disease (for example an immune-related disease and/or inflammatory disease).

As used herein “pharmaceutically acceptable carrier” or excipient includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier may be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media is incompatible with the active agent, use thereof in the pharmaceutical compositions of the invention is contemplated. Supplementary active compounds may also be incorporated into the compositions.

The present invention further relates to a method for blocking the interaction between CD40L and α5β1 integrin; the method may comprise the step of administering (to a subject) an effective amount of an agent that block the interaction between CD40L and α5β1. In an embodiment of the present invention, the interaction may occur at the cell surface and a method for blocking the cell surface interaction between CD40L and α5β1 integrin may comprise contacting the cell with an effective amount of an agent.

“Administration” of a composition may be performed by any suitable routes. Such routes may include parenteral, pulmonary, nasal and/or oral routes. In one embodiment, the pharmaceutical composition may be intra-muscular (IM), subcutaneous (SC), intra-dermal (ID), intra-venous (IV) and/or intra-peritoneal (IP) routes using any suitable means.

The term “effective amount” is intended to mean an amount of an agent sufficient to substantially block the interaction between CD40L and α5β1. An effective amount may also encompass either “therapeutically effective amount” and/or “prophylactically effective amount”. A “therapeutically effective amount” refers to an amount effective, at dosages and for periods of time necessary, to achieve the desired therapeutic result, such as a reduction in disease progression and/or alleviation of the symptoms associated with a disease. A therapeutically effective amount of modulators of CD40L activity may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. A “prophylactically effective amount” refers to an amount effective, at dosages and for periods of time necessary, to achieve the desired prophylactic result, such as preventing and/or inhibiting (reducing) the rate of disease onset or progression. A prophylactically effective amount may be determined as described above for the therapeutically effective amount. For any particular subject, specific dosage regimens may be adjusted over time according to the individual need and the professional judgment of the person administering of the compositions.

In a further aspect, the present invention provides a method of inhibiting production of an inflammatory mediator (by a cell), the method comprising blocking the interaction between CD40L and α5β1 integrin.

During inflammation, various molecules may be secreted by cells. Such molecules may be referred to as “inflammatory mediators”. As will be appreciated to one skilled in the art, these inflammatory mediators may be, for example and without limitation, amines, eicosanoids, growth factors, reactive oxygen species, enzymes (for example a proteinase), chemokines, cytokines, etc. In an embodiment of the present invention, the inflammatory mediator may be IL-6, IL-8 and/or a metalloproteinase. In a further embodiment, the metalloproteinase may be MMP-1, MMP-2 and/or MMP-9. For example, inhibition of metalloproteinase production may be particularly useful in diseases/conditions where the production/activation of metalloproteinases has undesirable effects.

In yet another aspect, the present invention relates to the use of an agent that may block the interaction between CD40L to α5β1 integrin and/or its use for the preparation of a medicament that may block the interaction between CD40L and α5β1 integrin.

In a further aspect, the present invention relates to the use of an agent for treating an inflammatory disease in a subject and/or for the preparation of a medicament for treating an inflammatory disease in a subject.

In an embodiment, the subject is a mammal, in a further embodiment, a human.

Given the correlation between CD40L-mediated α5β1 activity and the production of inflammatory mediators, agents capable of inhibiting the CD40L-mediated activation of α5β1 may be used for the prevention and treatment of inflammation-related disorders.

The present invention also further relates to screening methods for the identification and characterization of compounds capable of blocking the interaction between CD40L and α5β1 and/or the CD40L-mediated activation of α5β1.

The above-mentioned compounds may be used for prevention and/or treatment of inflammation-related diseases or conditions, or may be used as lead compounds for the development and testing of additional compounds having improved specificity, efficacy and/or pharmacological (e.g. pharmacokinetic) properties. In an embodiment the compound may be a prodrug which is altered into its active form at the appropriate site of action. In certain embodiments, one or a plurality of the steps of the screening/testing methods of the invention may be automated.

A method of identifying a compound capable of blocking the interaction between CD40L and α5β1 integrin method may comprise measuring the binding of CD40L to α5β1 integrin in the presence versus the absence of an agent, wherein a lower binding of CD40L to α5β1 integrin in the presence of the agent may be indicative that the agent is capable of blocking the interaction between CD40L and α5β1 integrin.

The methods for identifying a compound (screening method) mentioned herein may be employed either with a single test compound or a plurality or library (e.g. a combinatorial library) of test compounds. In the latter case, synergistic effects provided by combinations of compounds may also be identified and characterized.

Measuring the binding of CD40L to α5β1 integrin may be performed using (without limitation) such suitable assays as quantitative comparisons comparing kinetic and equilibrium binding constants. The kinetic association rate (kₒₙ) and dissociation rate (k_{off}), and the equilibrium binding constants (K_{d}) may be determined using surface plasmon resonance on a BlAcore™ instrument following the standard procedure in the literature. Binding properties of these interactions may also be assessed by flow cytometry (as described in Example 2 below) and/or by solid phase binding assay (as described in Example 3 below).

The present invention also relates to a method of identifying a compound capable of blocking the interaction between CD40L and α5β1 integrin; the method may comprise measuring a CD40L-mediated α5β1 integrin activity in the presence or absence of the agent, wherein a lower α5β1 integrin activity in the presence of the agent may be indicative that the agent is blocking the interaction between CD40L and α5β1 integrin.

As used herein, “an activity mediated by CD40L” or “CD40L-mediated α5β1 integrin activity” is an activity involving or resulting from the binding of CD40L to α5β1, and includes, but is not limited to, binding to α5β1, the association of α5β1 with the cytoskeleton, the activation of intracellular signaling molecules (for example Jun-N-terminal Kinase (JNK), members of the MAP kinase (MAPK) such as p38 and the phosphorylation of ERK1/2 or ERK pathways such as ERK1/2), the induction of T cells to produce and secrete cytokines (for example IL-2, IL-10, IFN-γ and TNF-α), the synthesis of inflammatory molecules (inflammatory mediators) such as IL-6, IL-8 and metalloproteinases and the mediation of platelet activation and/or aggregation.

In an aspect, the present invention relates to a method for identifying a compound capable of inhibiting or decreasing inflammation; the method may comprise measuring the binding of CD40L to α5β1 integrin in the presence versus the absence of the agent. A lower binding of CD40L to α5β1 integrin in the presence of the agent may be indicative that the agent is capable of inhibiting or decreasing inflammation.

In a further aspect, the present invention provides a method of identifying a compound capable of inhibiting or decreasing inflammation; the method may comprise measuring a CD40L-mediated α5β1 integrin activity in the presence versus the absence of the agent, wherein a lower α5β1 integrin activity in the presence of the agent may be indicative that the agent is capable of inhibiting or decreasing inflammation.

In a further aspect, the present invention provides a method of treating an inflammatory disease or condition in a subject; the method may comprise blocking the interaction between CD40L and α5β1 integrin in the subject.

In various embodiments, agents blocking the interaction between CD40L and α5β1 integrin may be used therapeutically in formulations or medicaments to prevent or treat CD40L-related disorders. CD40L-related disorders generally relate to various immune-mediated and/or inflammatory-related diseases/conditions. The modulators of CD40L activity may find use in disease conditions for which antagonism of immune cell activation, and more particularly CD40L-mediated immune activation, is desirable, including a variety of inflammatory and autoimmune diseases. Such diseases include, but are not limited to: systemic lupus erythematosus (SLE), arthritis, psoriasis, multiple sclerosis, allergic encephalitis, Crohn's disease, diabetes, Hodgkin's and non-Hodgkin's Lymphomas (NHL), chronic renal failure, nephrotic syndrome, mixed connective tissue disease, Hashimoto's thyroiditis, sickle cell anemia, inflammatory bowel disease, Hodgkin's disease, rheumatoid vasculitis, chronic lymphocytic leukaemia, myasthenia gravis, preeclampsia and cardiovascular conditions including atherosclerosis, thrombocytopenia (Purpura) and thrombosis.

Also, the modulators of CD40L activity (agents) have potential utility for treatment of other CD40L-related disorders such as non-autoimmune conditions wherein immunomodulation is desirable, e.g., graft-versus-host disease (GVHD), transplant rejection, asthma, bronchial hyperreactivity, leukemia, lymphoma, among others. Inflammation-related conditions contemplated for treatment in accordance with the present invention include arthritic diseases/conditions (e. g. rheumatoid arthritis, gouty arthritis, osteoarthritis, juvenile arthritis, systemic lupus erythematosus, spondyloarthopathies, and the like). Arthritic diseases/conditions including diseases such as rheumatoid arthritis, osteoarthritis and traumatic arthritis are inflammatory diseases that cause destruction of cartilage and bone mediated by inflammation of joint synovial membrane. Vascular neogenesis, lymphocyte invasion and proliferation and activation of synovial cells are observed in the inflamed synovial membrane. Activated synovial cells produce chemical mediators such as cytokines, prostaglandins and matrix metalloproteinases, and are considered to cause destruction of cartilage and bone, leading to joint inflammation (16, 17).

In a further aspect, the present invention provides a use of an agent capable of blocking the interaction between CD40L and α5β1 integrin for treating an inflammatory disease or condition in a subject.

In a further aspect, the present invention provides a use of an agent capable of blocking the interaction between CD40L and α5β1 integrin for the preparation of a medicament for treating an inflammatory disease or condition in a subject.

In a further aspect, the present invention provides a composition for treating an inflammatory disease or condition in a subject comprising an agent capable of blocking the interaction between CD40L and α5β1 integrin and a pharmaceutically acceptable carrier.

In a further aspect, the present invention provides a method of identifying a compound capable of controlling immune activation and inflammation without inducing a blood-related disorder, the method comprising:
(a) measuring a first binding or activity of CD40L to CD40 in the presence versus the absence of the agent;
(b) measuring a second binding or activity of CD40L to α5β1 integrin in the presence versus the absence of the agent,
(c) measuring platelet aggregation or activation in the presence of the agent;
   wherein a lower first and second binding or activity and a lower platelet activation or aggregation in the presence of the agent may be indicative that the agent is capable of controlling immune activation and inflammation without inducing a blood-related disorder.

“Bleeding disorders or complications” as used herein refers to any adverse effect on blood levels and physiology, including abnormality in any part of the system that controls bleeding (hemostasis) can lead to excessive bleeding or excessive clotting, such as platelet thrombotic responses, thrombophilia, thrombocytopenia, increased time to clot, increased bleeding time and blood loss. In an embodiment, the above-mentioned blood-related disorder may be bleeding or clotting disorder. In a further embodiment, the above-mentioned bleeding or clotting disorder may be thrombosis.

In a further aspect, the present invention provides a package comprising:
(a) an agent capable of blocking the interaction between CD40L and α5β1 integrin; and
(b) instructions for its use.

In an embodiment the use may be for the treatment or prevention of inflammatory-related diseases or condition in the subject.

Although various embodiments of the invention are disclosed herein, many adaptations and modifications may be made within the scope of the invention in accordance with the common general knowledge of those skilled in the relevant art. Such modifications include the substitution of known equivalents for any aspect of the invention in order to achieve the same result in substantially the same way. Furthermore, numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### Examples

### Example 1

### Materials and Methods

Cells. The myelomonocytic cell line U937 (ATCC, Manassas, Va., USA) and the B cell lymphoma cell line BJAB (obtained from Dr, J. Menezes, Sainte-Justine Hospital, Montréal, QC, Canada; (68) were maintained in RPMI 1640 containing 10% heat-inactivated FBS, L-glutamine, penicillin, and streptomycin (Wisent, St-Bruno, QC, Canada).

Reagents and antibodies. Recombinant trimeric soluble CD40L (sCD40L) (69) was provided by Immunex Corp. (Seattle, Oreg., USA). Avidin was purchased from Sigma (Sigma, St. Louis, Mo., USA). Alexa Fluor-488 labeling of rsCD40L (rsCD40L-A) and avidin (Avidin-A) was performed according to the manufacturer's instructions (Molecular Probes, Eugene, Oreg., USA). Anti-CD40L hybridoma 5C8 (IgG2a) and anti-CD40 hybridoma G28.5 (IgG1) were obtained from ATCC. The isotype controls anti-TSST-1 mAb 2H8 (IgG1) and anti-SEB mAb 8C12 (IgG2a) were developed in our laboratory. Anti-α5β1 mAb HA5 (IgG2b, commercially available at Chemicon, catalog #MAB1999) and isotype control mouse mAb IgG2b were provided by Dr. Bosco Chan (Roberts Research Institute, London, ON, Canada) (70). Anti-β1 mAb B44 has been previously described (71). The following antibodies were purchased: rabbit anti-α5 antibody (Chemicon, Temecula, Calif., USA), anti-α5 mAb P1D6 (IgG3) (Biomeda, Foster City, Calif., USA), and isotype control IgG3 anti-bacterial peptidoglycan MAB983 (Chemicon), goat anti-mouse IgG-FITC antibody (Sigma), rabbit anti-phospho-ERK1/2 and anti-ERK1/2 antibodies (Cell Signaling Technology, Inc., Beverly, Mass., USA), goat anti-rabbit IgG-HRP antibody and goat anti-mouse IgG-HRP antibody (Santa Cruz Biotechnology, Santa Cruz, Calif., USA), and anti-CD40 mAb 82102 (IgG1) (R & D Systems, Minneapolis, Minn., USA). Soluble α5β1 (sα5β1) was produced as previously described (72-73). Recombinant soluble CD40-Fc was from R & D Systems.

Plasmids and mutagenesis. pMTBipN5-His-(A) vector was purchased from Invitrogen Inc. pMTBipN5-His-(A)-hsCD40Lwt (human soluble CD40L wild type) was generated by subcloning sCD40Lwt at Bglll and Xhol in pMTBip/V5-His-(A) vector. Prior to the cloning, the 450 bp sequence encoding the soluble form of wild type CD40L (333 bp to 783 bp; M113 to L261 SEQ ID NO.3) was first amplified by PCR from pSecTag2A/CD40L (gift from Dr Daniel Jung) using a 5′ and 3′ sCD40L primers containing respectively a Bglll and Xhol restriction site (5′sCD40L-Bglll: 5′ CGGGAGATCTATGCAAAAAGGTGAT3′ SEQ ID NO.13, 3′sCD40L-Xhol:5′ TAGACTCGAGTTTGAGTAAGCC 3′ SEQ ID NO.14).

Murine sCD40Lwt was generated from pCDNA3.1-mCD40L-Lz (a gift from Dr Réjean Lapointe). pCDNA3.1-mCD40L-Lz contains sequence encoding extracellular domain of murine CD40L (residues D51 to L260). As described above for soluble hCD40L, primers with Bglll and Xhol (5′smCD40L-Bglll:5′CGGGAGATCTGATAAGGTCGAAG3′ SEQ ID NO.15 and 3′sCD40L-Xhol:5′ TAGACTCGAGTTTGAGTAAGCC 3′ SEQ ID NO.14) were used to amplify sequence encoding for soluble mCD40Lwt and subsequently for cloning in pMTBip/V5-His-(A).

Human CD40L SEQ ID NO.1 shares high homology with the murine form of CD40L SEQ ID NO.7. To generate monomeric forms of both murine and human sCD40L singles and doubles mutants of soluble CD40L were constructed using PCR-overlap method (84). All mutants and their primers are listed in Table 2 and 3. Briefly, a 5′ PCR product was generated from pMTBipN5-His-(A)-sCD40Lwt using the 5′sCD40L-Bglll primer describe above and a reverse mutagenic primer (see Table 2 and 3). A 3′PCR product was also generated, using a forward complementary mutagenic primer and the 3′sCD40L-Xhol. The two overlapping PCR products were mixed and a final PCR was performed using the 5′ and 3′sCD40L external primers. The PCR product was then subcloned in pMTBip/V5-His-(A) at Bglll and Xhol. The four plasmid constructs pMTBip/V5-His-(A)-hsCD40LY170A, H224A, G226A and pMTBip/V5-His-(A)G227A and the four murine equivalents were then sequenced to confirm mutation. To generate human sCD40L double mutant Y170/H224 SEQ ID NO.40, Y170/G226 SEQ ID ID NO.41, Y170/G227 SEQ ID NO.6, plasmid construct of Y170 single mutant was used as template with the different mutagenic primers for H224, G226 and G227 as described for generating single mutant. Murine double mutant were also generate in the same manner. All double mutant constructs were also sequenced to confirm mutation.

TABLE 2
PRIMERS FOR GENERATING MONOMERIC FORM OF
SOLUBLE hCD40L

| MUTANT | PRIMERS | | |
|---|---|---|---|
| Y170A | CD40L-Y170A-C | 5′ caa gga ctc | SEQ ID NO. 16 |
| | | tat gct atc tat | |
| | | gcc 3′ | |
| | CD40L-Y170A-B | 5′ ggc ata gat | SEQ ID NO. 17 |
| | | agc ata gag tcc | |
| | | ttg 3′ | |
| H224A | CD40L-H224A-C | 5′ caa caa tcc | SEQ ID NO. 18 |
| | | att gca ttg gga | |
| | | gga g 3′ | |
| | CD40L-H224A-B | 5′ c tcc tcc caa | SEQ ID NO. 19 |
| | | tgc aat gga ttg | |
| | | ttg 3′ | |
| G226A | CD40L-G226A-C | 5′ c att cac ttg | SEQ ID NO.20 |
| | | gca gga gta ttt | |
| | | g 3′ | |
| | CD40L-G226A-B | 5′ c aaa tac tcc | SEQ ID NO. 21 |
| | | tgc caa gtg aat | |
| | | g 3′ | |
| G227A | CD40L-G227A-C | 5′ cac ttg gga | SEQ ID NO. 22 |
| | | gca gta ttt gaa | |
| | | ttg 3′ | |
| | CD40L-G227A-B | 5′ caa ttc aaa | SEQ ID NO. 23 |
| | | tac tgc tcc caa | |
| | | gtg 3′ | |

TABLE 3
PRIMERS FOR GENERATING MONOMERIC FORM OF
SOLUBLE mCD40L

| MUTANT | PRIMERS | | |
|---|---|---|---|
| Y169A | CD40L-Y169A-C | 5′ gga ctc tat | SEQ ID NO 24 |
| | | gct gtc tac act | |
| | | c 3′ | |
| | CD40L-Y169A-B | 5′ gag tgt aga | SEQ ID NO. 25 |
| | | cag cat aga gtc | |
| | | c 3′ | |
| H223A | CD40L-H223A-C | 5′ cag tct gtt | SEQ ID NO. 26 |
| | | gca ttg ggc | |
| | | gg 3′ | |
| | CD40L-H223A-B | 5′ ccg ccc aag | SEQ ID NO. 27 |
| | | tca aca gac | |
| | | tg 3′ | |
| G225A | CD40L-G225A-C | 5′ gtc tgt tca | SEQ ID NO. 28 |
| | | ctt ggc cgg agt | |
| | | g 3′ | |
| | CD40L-G225A-B | 5′ cac tcc ggc | SEQ ID NO. 29 |
| | | caa gtg aac aga | |
| | | c 3′ | |
| G226A | CD40L-G226A-C | 5′ gtt cac ttg | SEQ ID NO. 30 |
| | | ggc gca gtg ttt | |
| | | g 3′ | |
| | CD40L-G226A-B | 5′ caa aca ctg | SEQ ID NO. 31 |
| | | cgc cca agt gaa | |
| | | c 3′ | |

Cell culture, transfection and protein purification. Schneider's (S2) cells were cultured in complete DES Schneider's Drosophila medium (Invitrogen Corp.), supplemented with 10% heat-inactivated Fetal Bovine Serum (F.B.S) (Wisent Inc.), 100 Units of Penicillin G Sodium, 100 mg/ml of Streptomycin Sulfate and 0.25 mg/ml of amphotericin B as fungicide (Invitrogen Corp.) at a cell density between 1.5-3×10⁶ cells/ml at 26° C. without CO₂. To produce recombinant soluble CD40L protein with different point mutations, cells were co-transfected with pMT-BiPN5-6× HisA carrying sCD40L DNA sequence and pCoHygro vectors (Invitrogen Corp.) at a weight ratio of 19:1, respectively, by the method of calcium phosphate. The clones were then selected with a final concentration of 300 mg/ml of hygromycin B (Wisent Inc.). The expression of recombinant sCD40L proteins were tested by seeding 1-2×10⁶ cells in 100 mm petri dish followed by induction with 100 mM of copper sulfate for 24 hours, cell debris were removed and further Western blot analysis of supernatant generated were performed. A stable population of hygromycin resistant S2 cells was obtained after 20 days period. The selected cultures were initiated to a large scale-up in Drosophila-SFM serum free medium (Invitrogen Corp.) supplemented with antibiotics-antimycotic (Invitrogen Corp.) without addition of hygromycin B. Upon induction, the proteins remaining in the supernatant were recovered by centrifugation of supernatant at 3000 g for 20 min. The samples were filtered through a 0.22 mm filter and concentrated to 1/20 initial volume with Centricon (Amicon 8400) concentrator. The hexahistidine tag from pMT-Bip/V5-6× HisA vector present in the recombinant protein was used to purify sCD40L proteins on HisTrap-HP column (Amersham-Pharmacia Biotech).

Flow cytometric analysis. For the sCD40L-A binding assay, cells were incubated in binding assay medium (RPMI 1640, HEPES 10 mM, BSA 1%) containing 200 ng of rsCD40L-A or avidin-A/2×10⁵ cells/100 μl for 1 h at 37° C. in a humidified incubator and a 5% CO₂ atmosphere. For competition binding with mAbs directed against cell surface molecules (CD40 and α5β1), cells (pre-incubated with 10 μg human IgG/10⁶ cells in staining medium for 15 min at 37° C.) were incubated with mAb for 30 min at 37° C. prior to labeling with rsCD40L-A. For competition binding with sα5β1 or mAbs directed against soluble molecules, rsCD40L-A and avidin-A were incubated with sα5β1 or mAbs for 1 h at 37° C. prior to the addition of the cells. Cell surface analyses with mAbs were performed as previously described (74). Washed cells were analyzed on a FACSort™ (Becton-Dickinson, Mountain View, Calif., USA).

Integrin activation. Cells (10⁶/ml HBSS) were incubated with Mn²⁺ (1 mM in HBSS) or DTT (10 mM in HBSS) for 30 min at room temperature. The DTT-treated cells then were washed twice in HBSS and resuspended in HBSS. The Mn²⁺-stimulated cells were used without washing.

Cell binding assay. The wells of microtiter plates (Nunc Maxisorp, VWR International Ltd., Mississauga, ON, Canada) were coated with gelatin (30 mg/ml in PBS) for 2 h at 37° C. Unbound gelatin was removed and the wells were air-dried for 1 h at 37° C. Fibronectin (5 μg/ml in PBS, Chemicon) was added to the wells and the plates were incubated overnight at 4° C. The wells were washed with PBS and blocked with 1% BSA in PBS for 1 h at room temperature. Control wells were coated with gelatin and BSA. Cells (5×10⁴ cells/well in PBS) were added to the wells and the plates were incubated for 1 h at 37° C. Unbound cells were removed and the wells were washed three times with PBS under mild agitation. Bound cells were analyzed under a Zeiss microscope (Zeiss Axiovert 100 (Carl Zeiss, Inc., Thornwood, N.Y., USA)) and photographed with a 3-CCD Color video camera, model DXC-390P (Sony Electronics Inc., Park Ridge, N.J., USA). The images were analyzed with Northern Eclipse 6.0 software (Empix Imaging Inc., Mississauga, ON, Canada). The adherent cells were then fixed with 1% paraformaldehyde in PBS for 30 min at room temperature and stained with 0.5% crystal violet in 20% methanol. After thoroughly washing the wells with tap water, the cells were lysed with 1% SDS in water and the absorbance at 595 nm (Thermomax microplate reader, Molecular Devices, Sunnyvale, Calif., USA) was determined.

Solid phase binding assay. The wells of microtiter plates (Nunc Maxisorb) were coated with 4 μg/ml of purified sα5β1 or soluble recombinant CD40 in PBS (pH 7.5) (50 μl/well) overnight at room temperature. After three washes with PBS containing 0.05% Tween 20 (PBS-T), the wells were blocked with 0.5% BSA in PBS for 2 h at room temperature. After three washes with PBS-T, rsCD40L was added to the wells at the indicated concentration and the plate was incubated for 3 h at room temperature. The wells were washed three times with PBS-T and bound rsCD40L was detected using goat anti-CD40L-biotin (R & D Systems) (2 h, room temperature) and streptavidin-HRP (Sigma) (2 h, RT), and revealed with TMB substrate (Sigma).

Cell stimulation. U937 and BJAB cells were incubated in serum-free medium for 4 h at 37° C. and stimulated with rsCD40L (250 ng/5×10⁵ cells) for 5 and 15 min at 37° C. The stimulation was stopped by the addition of hot 2× SDS sample buffer containing 10% 2-mercaptoethanol (2-ME), protease inhibitors (Roche, Montreal, QC, Canada) and phosphatase inhibitors (Sigma). After boiling for 7 min, cell lysates were separated by SDS-PAGE for Western blot analysis.

Receptor translocation to the cytoskeleton. U937 and BJAB cells were stimulated with rsCD40L (250 ng/5×10⁵ cells) for 30 min at 37° C. in binding assay medium and washed three times in PBS. Cells were lysed in Triton™ X-100 buffer (25 mM Tris-HCl, pH 7.4, 150 mM NaCl, 5 mM EDTA, 1× complete protease inhibitor cocktail (Roche), and 1% Triton™ X-100 (Fisher Scientific, Montreal, QC, Canada) for 30 min on ice. The cell lysates were centrifuged at 16,000×g for 15 min at 4° C. Soluble and insoluble fractions were separated by SDS-PAGE under non-reducing conditions and analyzed by immunoblotting.

Immunoblot analysis. PVDF membranes were blocked in blotto (5% skim milk in Tris saline pH 7.5, 0.15% Tween 20 (Fisher Scientific) for 1 h at room temperature, and incubated with rabbit anti-α5 antibody overnight at 4° C. followed by goat anti-rabbit IgG-HRP antibody or with mouse anti-CD40 antibody overnight at 4° C. followed by goat anti-mouse IgG-HRP antibody. The phosphorylation of ERK1/2 was assessed by immunoblotting using phospho-specific Abs according to the manufacturer's instructions. Membranes were stripped (62 mM Tris-HCl pH 6.8, 2% SDS/100 mM, 2-ME, 30 min, 50° C.) and reprobed with antibody recognizing total ERK1/2. Antigen-antibody complexes were revealed with ECL (GE Healthcare, Mississauga, ON, Canada).

Analysis of IL-8 mRNA expression. U937 cells (2×10⁶/100 μ) were treated with 100 ng of rsCD40L in RPMI 5% FBS at 37° C. for the indicated time points. Reactions were stopped by adding ice-cold RPMI, and cells were isolated by spinning at 14000 rpm at 4° C. Total RNA was prepared from each sample using the Rneasy total RNA isolation Kit (Qiagen Inc., Mississauga, ON, Canada). Single-strand cDNA for a PCR template was synthesized from 1 μg of total RNA using a primer, oligo(dT)₁₂₋₁₈ (Invitrogen, Burlington, ON, Canada) and superscript III reverse transcriptase (Invitrogen) under the conditions indicated by the manufacturer. Reverse transcription was inactivated at 95° C. for 5 min and the products were kept on ice until needed for the PCR. Specific primers were designed from cDNA sequence for IL-8 and p-actin. Each cDNA was amplified by PCR using Taq DNA polymerase (Invitrogen). The sequences of the primers were as follows: IL-8F (5′GCCAAGGAGTGCTAAAGAAC-3′) SEQ ID NO.32, IL-8R (5′-CACTGGCATCTTCACTGATTCTTG-3′) SEQ ID NO.33, 13-actin F (5′-AATCTGGCACCACACCTTCT-3′) SEQ ID NO.34, β-actin R (⁵′-TAATGTCACGCACGATTTCC-3′) SEQ ID NO.35. Conditions for PCR were 35 cycles of 94° C. for 45 s, 55° C. for 45 s, 72° C. for 1 min. Additional 10 min of 72° C. was performed at the end of PCR reaction. The products were analysed on a 1% agarose gel containing ethidium bromide. The expected sizes of the PCR products for IL-8 and β-actin were 280 base pairs (bp), and 400 bp, respectively. We did not detect any band when we performed PCR without adding the cDNA template in this study. Genomic DNA contaminants were examined by performing PCR reaction on 1 μg of total RNA at similar conditions, and no contaminants were detected. Densitometric analyses were performed on each detected band using a Molecular Imager Gel Doc System and Quantity One analysis software from Bio-Rad (Bio-Rad Laboratories, Mississauga, ON, Canada). Results shown are normalized for two conditions, first based on β-actin levels at each time points and after, based on the level of expression of each gene of samples indicated as time 0 (non-treated samples). The ratio was then blotted as fold increase of IL-8 mRNA after rsCD40L treatment versus time of the treatment.

in vivo thrombosis model. An in vivo approach to assess thrombosis in the presence of CD40L modulators was developed. First, the carotid artery (right) of a C57BL/6 mouse, while being monitored for its blood flow using a miniature flow probe, received a FeCl₃ stimulation inducing thrombus formation. In the treatment group, mice receive, 10 min prior to the FeCl₃ injury, an IV injection of the treatment (CD40L interfering agents and/or molecules). The control group receives saline prior to the injury. At the end of all experiments blood flow amplitude is assessed.

Bronchial fibroblasts/T cell co-cultures and IL-6 measurements. Fibroblasts were isolated from bronchial biopsies obtained in healthy or mild asthmatic patients. Following isolation, biopsies were immediately treated overnight at 4° C. with 500 μg/mL of thermolysine (Sigma Chemical Co., St. Louis, Mo.) allowing complete separation from the epithelium. Remaining tissues were subsequently treated with (0.1%) collagenase (Roche, Laval, QC). Fibroblasts were identified and characterized by immunofluorescence and FACS analysis using anti-vimentin as well as an antibody directed against the fibroblast specific Ab-1 antigen (Calbiochem, San Diego, Calif.). Fibroblasts were then let to adhere overnight in 1 mL of DMEM at 37° C. and 5% CO2. For T cell preparation, peripheral blood was subjected to gradient separation using Ficoll-Paque (GE Healthcare Bio-Sciences) which allow the retrieval of lymphocytes and monocytes. These cells were resuspended in RPMI media and incubated for 1 h at 37° C. and 5% de CO2 thereby allowing adherence of monocytes. Lymphocyte were recuperated by passing them through a nylon column (Polysciences Inc., Warrington, Pa.) allowing B cells to adhere and recuperating T cells. Isolated cells were let to interact using 5×106 cells fibroblasts and 5×10⁵ T cells for 6 h. Following interaction, mRNA was extracted from fibroblasts and IL-6 measured by real time PCR using the following primers: IL-6 Sens 5′-TCT CCA CAA GCG CCT TCG-3′ (SEQ ID NO.36) and IL-6 anti-sense 5′-CTG AGG GCT GAG ATG CCG-3′ (SEQ ID NO.37) as well as an hybridization temperature of 60° C.

Bronchial hyperreactivity model. 5-6-week-old male Balb/c mice were sensitized to OVA (200 μg intransally) for ten days, and boosted with a single dose on day 22. They were challenged with the same dose of OVA on days 29 through 33. One hour before allergen challenge, 20 μg of anti-CD40 (clone FGK 45.5) or anti-CD40L (MR1) was administered intranasally. On day 34, bronchial hyperresponsiveness was assessed by inhaled methacholine challenge. Animals were anaesthetized, tracheotomized, paralyzed, and ventilated by the flexiVent small animal ventilator (SCIREQ, Montreal, Quebec). Pulmonary resistance measurements were taken at baseline, and following nebulised doses of 15.625, 31.25, 62.5, 125, and 250 μg/mL methacholine choride in normal saline.

Following respiratory mechanics measurements, animals were taken off the ventilator and sacrificed.

### Example 2

### rsCD40L-A Binds to CD40-Negative Cells

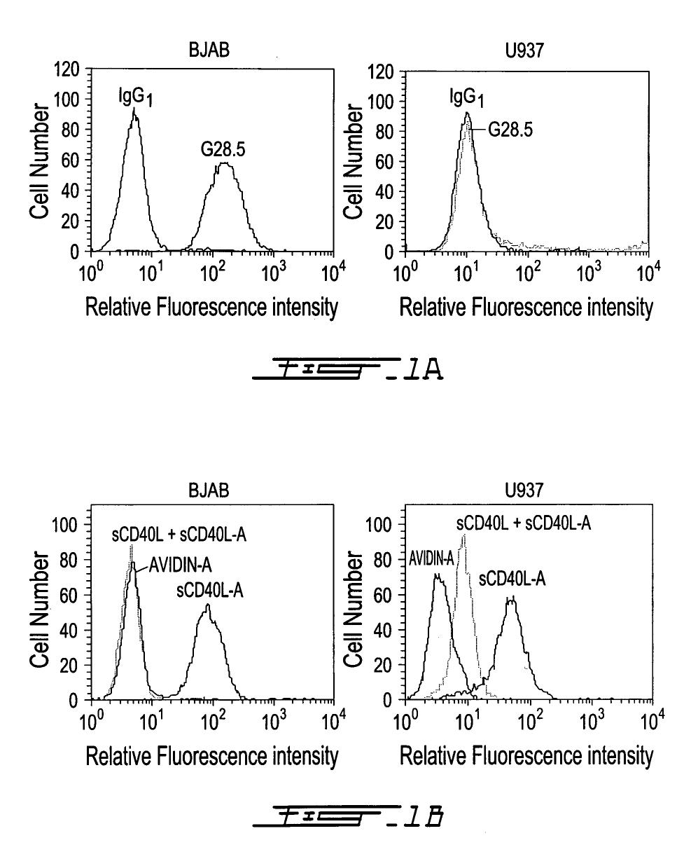

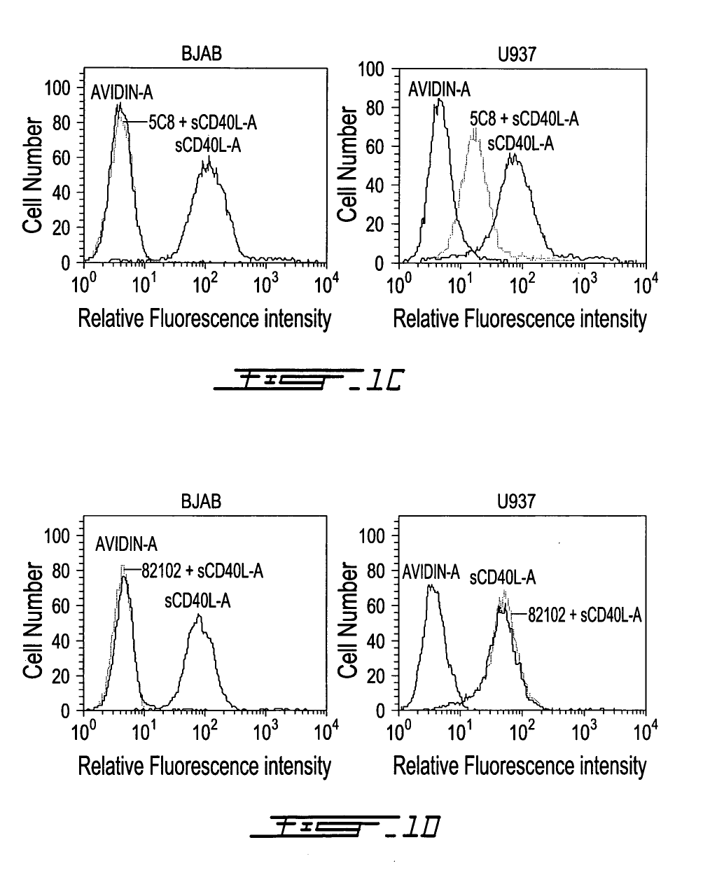

Expression of CD40 was analyzed by flow cytometry and immunoblotting using various cell lines, and CD40-negative cell lines that could bind Alexa Fluor-labeled rsCD40L (rsCD40L-A) were selected. The CD40-positive human BJAB B cell line was used as a control. The monocytic U937 cell line did not express CD40, as assessed by flow cytometry (FIG. 1A) and immunoblotting but was able to bind rsCD40L-A at a level similar to that observed on BJAB cells (FIG. 1B). The specificity of this binding was confirmed by adding 10-fold excess unlabelled rsCD40L (FIG. 1B) or by pre-incubating rsCD40L with the anti-CD40L mAb 5C8 (FIG. 1C). Similar results were obtained with the CD40-negative erythroleukemic K562 and HEK 293 cell lines. These results indicate that rsCD40L binding to U937 cells is CD40-independent. FIG. 1D shows that pre-incubation of U937 cells with blocking anti-CD40 mAb 82102 did not interfere with the binding of rsCD40L-A whereas the same treatment completely prevented the binding of rsCD40L-A to BJAB cells, indicating that U937 cells express at least one molecule that is distinct from CD40 and that acts as a receptor for sCD40L.

### Example 3

### rsCD40L Binds to α5β1

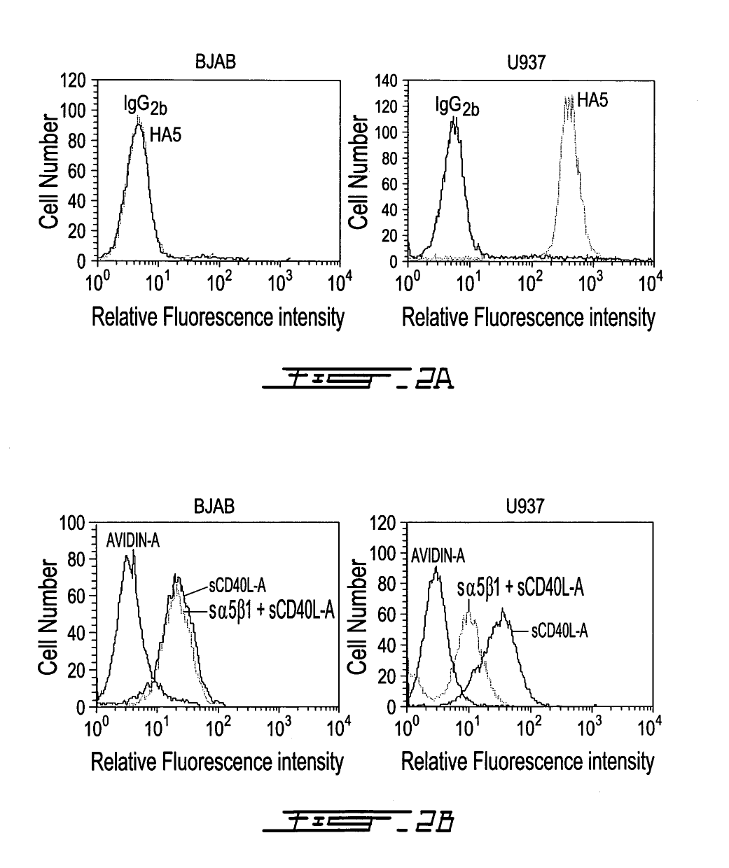

It was determined herein that α5β1 was expressed constitutively on U937 cells (FIG. 2A), K562 cells (75), and HEK 293 cells (76) but not on BJAB cells (FIG. 2A). To determine whether rsCD40L-A could bind to α5β1, we performed a competitive binding assay using soluble α5β1 (sα5β1) as bait for rsCD40L-A. FIG. 2B shows that pre-incubation of rsCD40L-A with sα5β1 substantially inhibited the binding of rsCD40L-A to U937 cells. Pre-incubation of rsCD40L-A with sα5β1 did not affect rsCD40L-A binding to CD40 on B cells, suggesting that sCD40L could bind concomitantly to both CD40 and α5β1.

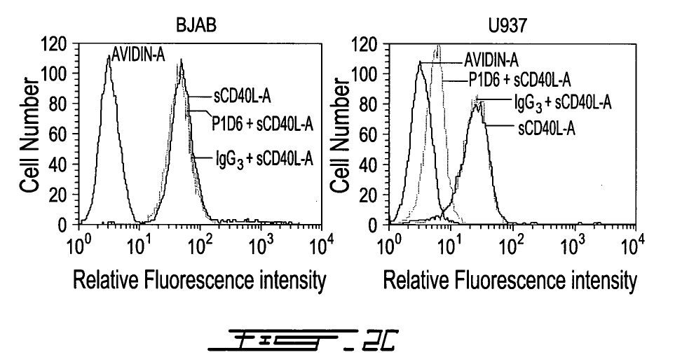

To further study that α5β1 is involved in the binding of rsCD40L to U937 cells, cells were pre-incubated with anti-α5 mAb P1 D6 or isotype control mAb and then incubated with rsCD40L-A. FIG. 2C shows that mAb P1 D6 inhibited the interaction of rsCD40L-A to U937 cells but did not affect the binding of rsCD40L-A to α5β1-negative BJAB cells. Thus, the results presented here indicate that α5β1 is a receptor for sCD40L. Lastly, to confirm that sCD40L binds directly to α5β1, a solid phase binding assay was developed using immobilized soluble CD40-Fc (sCD4O-Fc) as a control receptor (FIG. 3A). The results presented in FIG. 3B clearly demonstrate that rsCD40L binds directly to purified α5β1 in a dose-dependent manner. Thus, α5β1, like CD40 and αllbβ3, is a receptor for sCD40L.

### Example 4

### Chemical Agents that Increase the Affinity of α5β1 for Fibronectin Negatively Affect the Binding of rsCD40L to U937 Cells

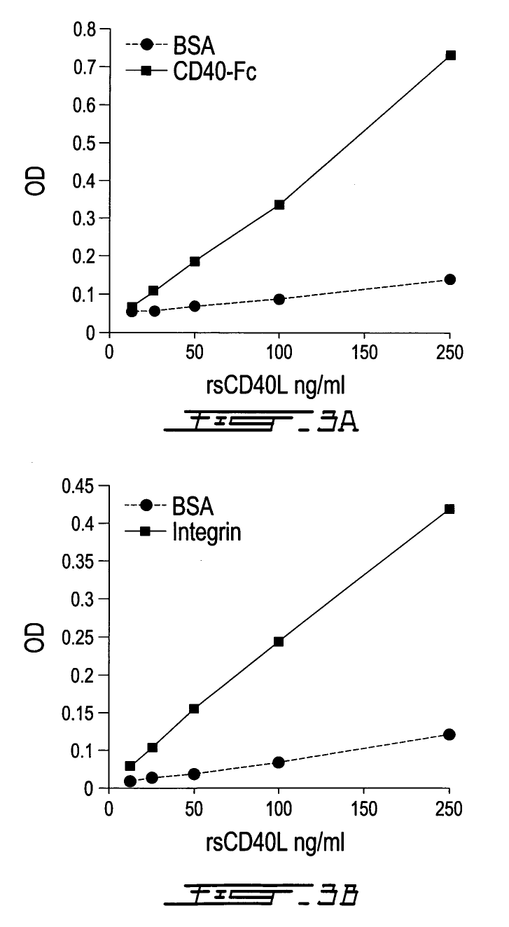

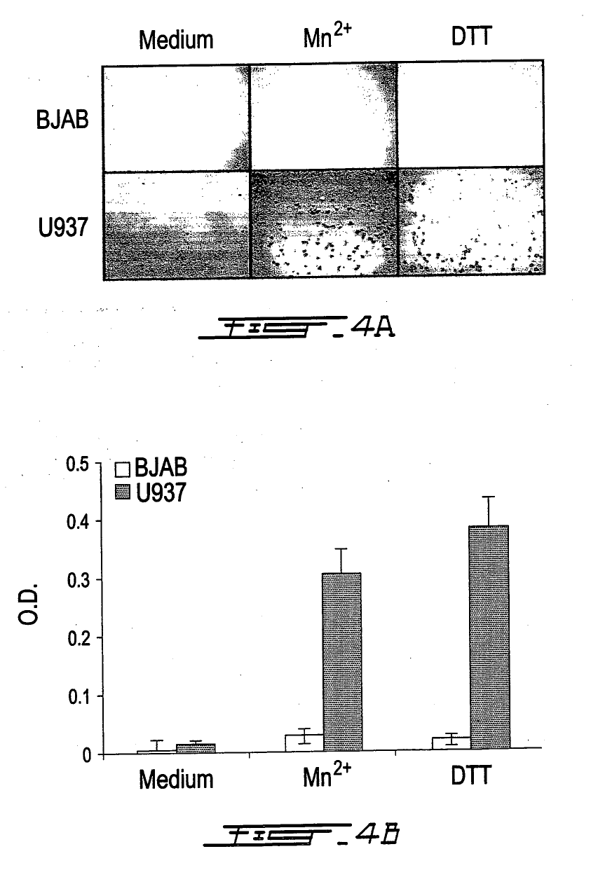

α5β1 is constitutively expressed on the cell surface in an inactive form that cannot bind fibronectin (reviewed in 77). Conformational changes triggered by outside-in or inside-out signaling result in the activation of the integrin (77) allowing it to bind to its natural ligand. Chemical agents such as Mn²⁺ and DTT can promote such changes (75). We wondered whether the activation of α5β1 integrin could also modulate the binding of rsCD40L to U937 cells. First, we confirmed that U937 cells did not constitutively bind to fibronectin and that Mn²⁺ and DTT strongly promoted their adhesion to fibronectin as evaluated by microscopy (FIG. 4A) and a colorimetric assay (FIG. 4B). In contrast, similar treatments of α5β1-negative BJAB cells did not promote their attachment to fibronectin (FIG. 4). Second, conformational changes induced by these chemical agents expose a β1 epitope, the mAb B44 epitope (Ni, H. et al., supra). Indeed, the results presented in FIG. 5A show that treatment with Mn²⁺ or DTT induced the expression of the B44 epitope on U937 cells but not on BJAB cells. We then assessed the binding of rsCD40L-A to U937 cells treated with Mn²⁺ or DTT. Interestingly, the treatment of U937 cells with Mn²⁺ reduced the binding of rsCD40L-A while the treatment with DTT almost completely inhibited the binding of rsCD40L-A to U937 cells (FIG. 5B). In contrast, similar treatments of BJAB cells had no effect on the binding of rsCD40L-A to CD40 (FIG. 5B). Thus, changes in the conformation of α5β1 that promote its binding to fibronectin decrease its interaction with sCD40L.

### Example 5

### rsCD40L Induces α5β1 Recruitment to the Cytoskeleton in CD40-Negative U937 Cells

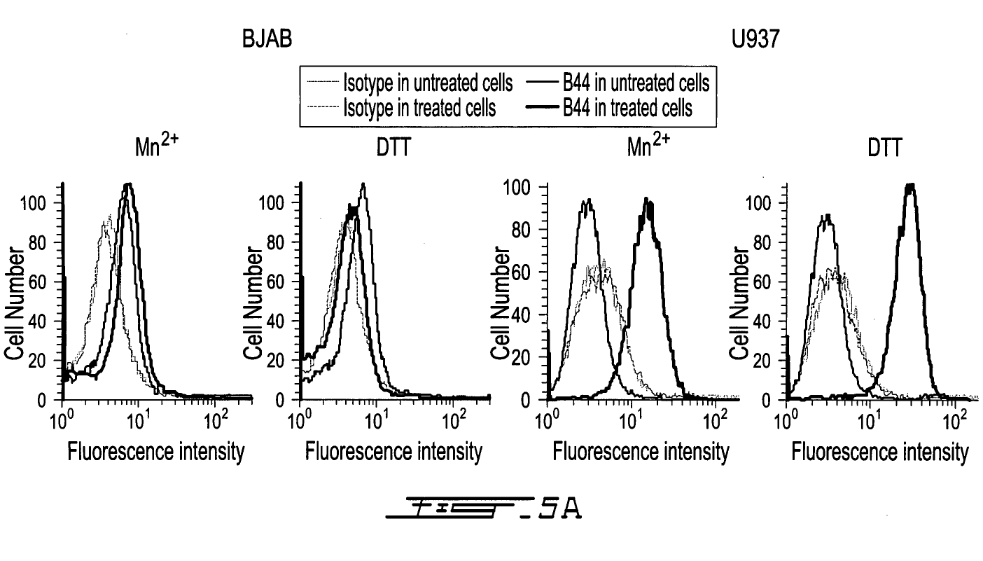

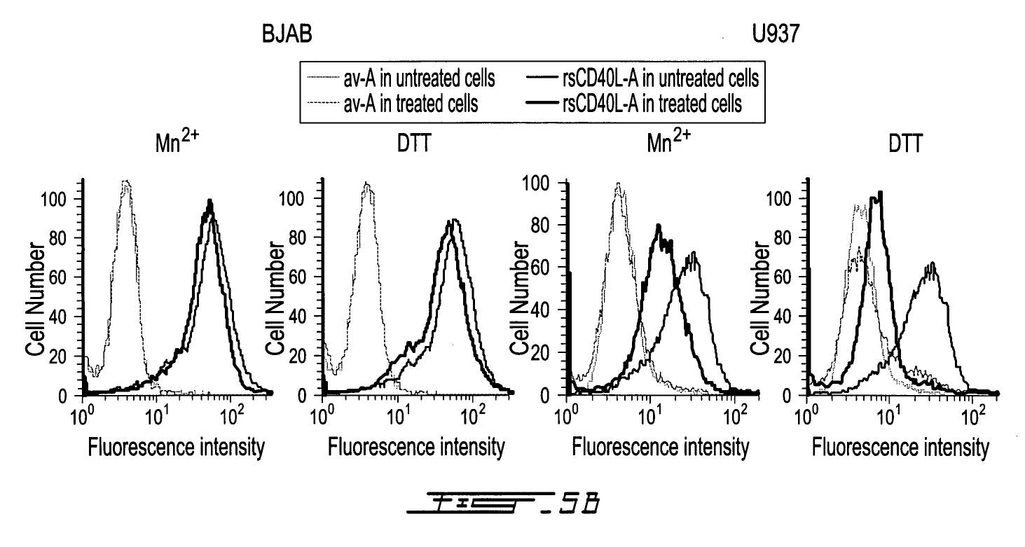

We studied whether the interaction of rsCD40L with α5β1 would result in its association with the cytoskeleton. To assess the recruitment of α5β1 to the cytoskeleton, cells were incubated with rsCD40L for 30 min at 37° C. and solubilized in Triton™ X-100 buffer. The soluble (Sol) and insoluble fractions (Ins) were separated by centrifugation and analyzed by immunoblotting with a rabbit polyclonal anti-α5 Ab (FIG. 6A). α5β1 was found exclusively in the Triton™ X-100-soluble fraction of unstimulated U937 cells whereas a significant amount of α5β1 translocated into the Triton™ X-100-insoluble fraction of rsCD40L-stimulated U937 cells. Also, rsCD40L induced the translocation of CD40 to the detergent-insoluble fraction of BJAB B cells (78), and the formation of CD40 homodimers (79) (FIG. 6B). Thus, the interaction of rsCD40L with α5β1 triggered its association with the cytoskeleton.

### Example 6

### rsCD40L Induces the Activation of the ERK1/2 Pathway in CD40-Negative U937 Cells

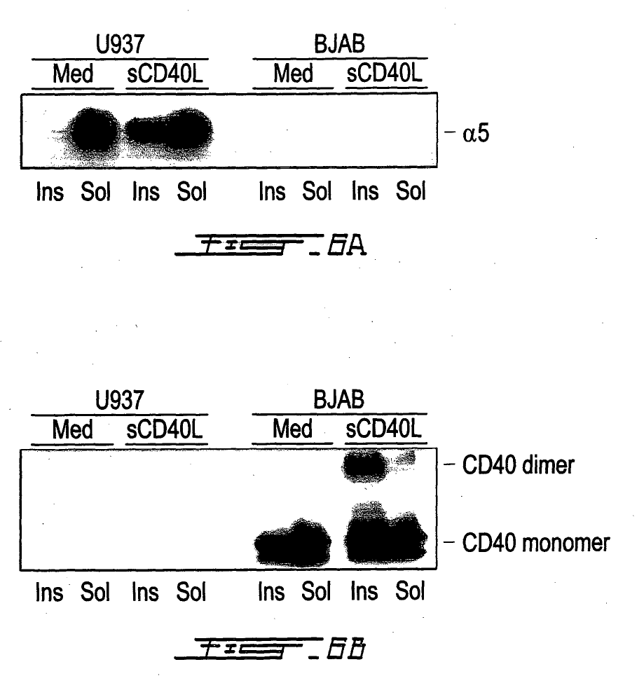

We then investigated signal-transducing events triggered by the engagement of α5β1 by rsCD40L. We thus investigated the phosphorylation of ERK1/2 in U937 cells following stimulation with rsCD40L and found, as shown in FIG. 7A, that ERK1/2 was rapidly phosphorylated in rsCD40L-activated U937 cells. The binding of rsCD40L to CD40 also induced the activation of ERK1/2 in BJAB cells (FIG. 7A). These results indicated that the interaction of sCD40L with α5β1 triggers the activation of signaling pathways, confirming the functional relevance of this interaction.

### Example 7

### The Interaction between sα5β1 and rsCD40L does not Interfere with the CD40L-Induced Activation of ERK1/2 via CD40

We showed above by flow cytometry analysis that purified sα5β1 prevented the interaction of rsCD40L with U937 cells. The interaction of rsCD40L with purified sα5β1 also completely prevented the activation of the ERK1/2 in U937 cells (FIG. 7B). An interesting outcome of the binding experiments was the observation that rsCD40L may interact concomitantly with CD40 and α5β1 (FIG. 2B). This suggested that sCD40L bound to α5β1 can trigger signaling in CD40-positive cells. Indeed, the data in FIG. 7B show that rsCD40L bound to sα5β1 induced the activation of ERK1/2 in BJAB cells. Thus, sCD40L may serve as a molecular bridge between CD40 and α5β1 expressed on two different cells and trigger signal transduction in both cells.

### Example 8

### Binding of CD40L to α5β1 Induces the Production of Inflammatory Molecules

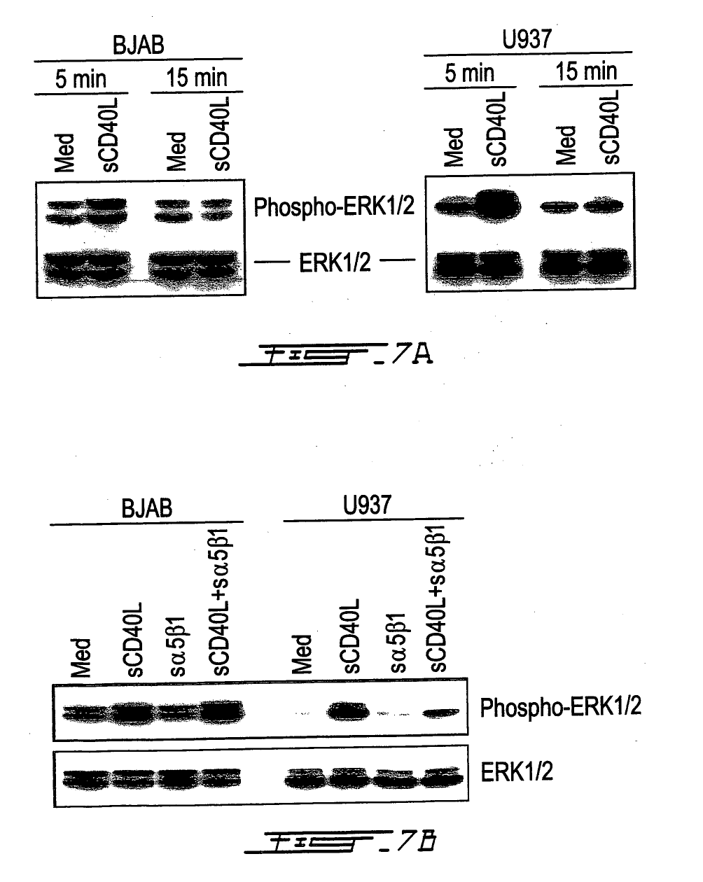

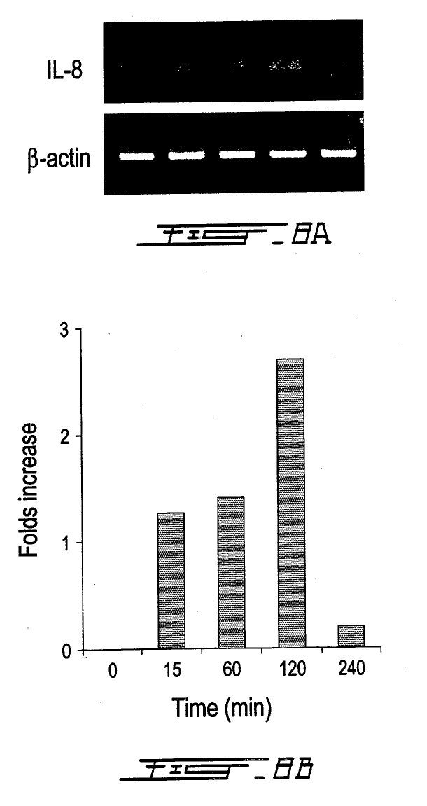

We then studied the binding of sCD40L to α5⊕1 induces the secretion of molecules by U937 cells. FIGS. 8 and 9 demonstrates that sCD40L induces the production of inflammatory-associated molecules such as IL-8 mRNA (FIG. 8) and metalloproteinases, namely MMP-2 and MMP-9 (FIG. 9A) as well as pro-MMP-1 (MMP-1 pro-enzyme) (FIG. 9B).

Findings presented above show that (1) sCD40L binds to α5β1 integrin, a widely distributed cell-surface receptor, (2) the interaction of sCD40L with α5β1 integrin is abrogated by conformational changes of α5β1 integrin that result in its activation and its binding to fibronectin, (3) binding of sCD40L to α5β1 integrin induces intracellular signaling and translocation of α5β1 integrin to the Triton™ X-100-insoluble fraction, (4) sCD40L may simultaneously bind to CD40 and α5β1 integrin on the cell surface and (5) CD40L induces the production of inflammatory molecules in cells expressing α5β1 integrin. Thus, α5β1 integrin is a functional receptor for sCD40L.

### Example 9

### Monomeric Soluble CD40L Bind ti their Receptors but Fail to Induce p38 Phosphorylation

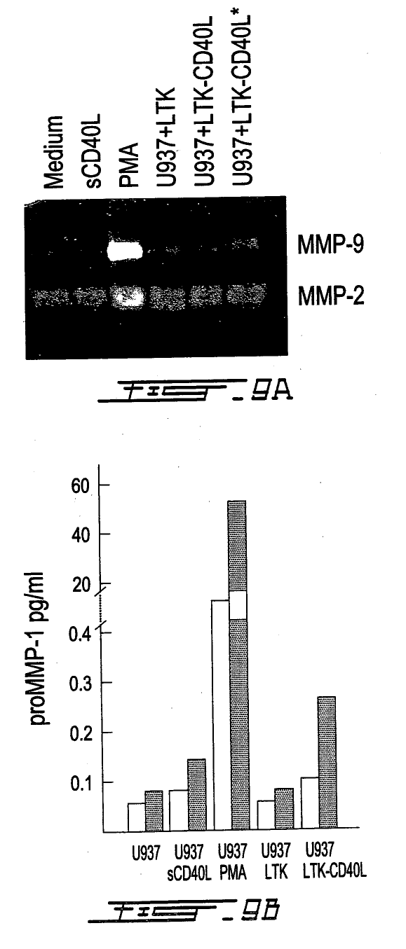

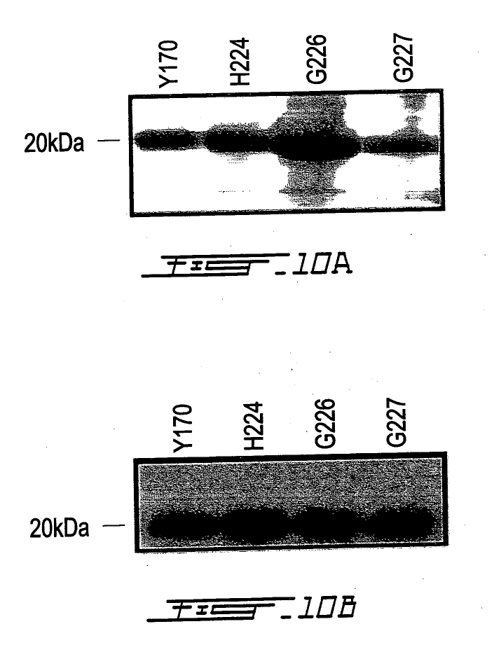

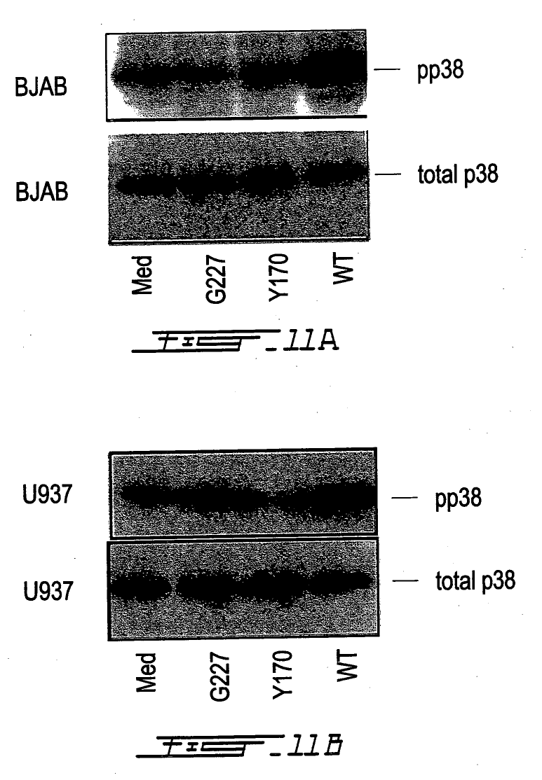

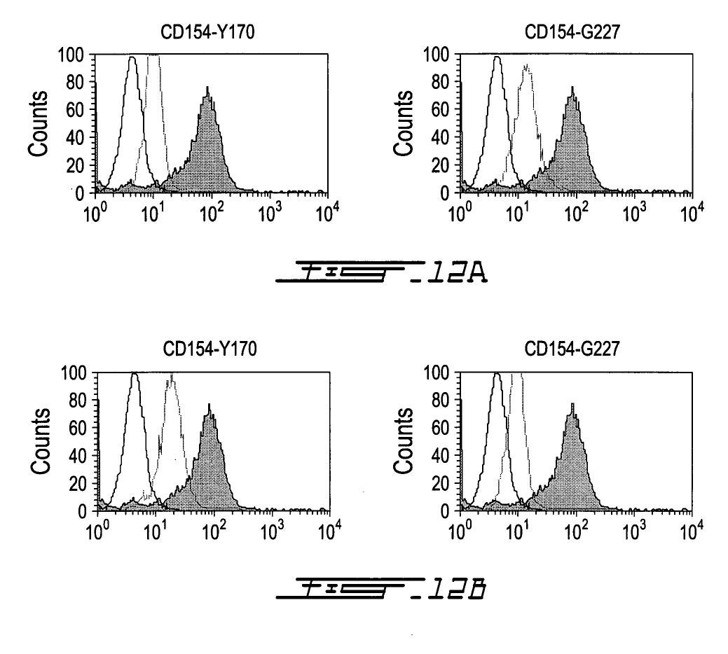

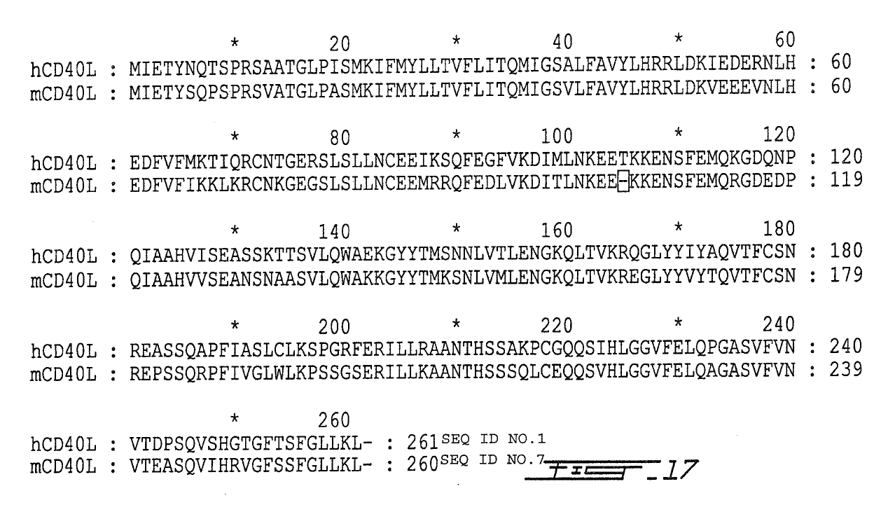

It is well established that CD40L is mainly expressed as a trimer when present at the cell surface and in soluble form (81). The trimer state is an absolute requirement for the biological activity of CD40L. By examining the crystal structure of CD40L (83), it was hypothesized that residues located at position Y170, H224, G226 and G227 in human CD40L sequence may be involved in trimer formation. Mutagenesis of each of these residues was performed by mutating them to alanine residues as described in Example 1. Recombinants were then generated, purified, and first analyzed for their purity by Commassie Blue and Western blot (FIG. 10). Two of these mutants, namely CD40L Y170 SEQ ID NO.4 and CD40L G227 SEQ ID NO.5 were tested for the induction of p38 phosphorylation in BJAB and U937 cells. The other two mutants generated (CD40L H224 SEQ ID NO.38 and CD40L G226 SEQ ID NO.39) were insoluble under biological conditions. FIG. 11 shows a significant decrease in the activity of CD40L Y170A SEQ ID NO.4 and CD40L G227A SEQ ID NO.5 mutants showing that these two residues are required for CD40L trimerization. Furthermore, these two mutants completely blocked the binding of Alexa-488 labeled sCD40L to BJAB and U937 cells (FIG. 12), supporting the monomeric status of these mutants and their ability to bind to their receptors. Murine soluble monomeric CD40L were also generated and are described in Example 1. A sequence alignment of hCD40L SEQ ID NO.1 and mCD40L SEQ ID NO.7 is shown in FIG. 17.

### Example 10

### Anti-CD40L Prevents Thrombosis in Mice

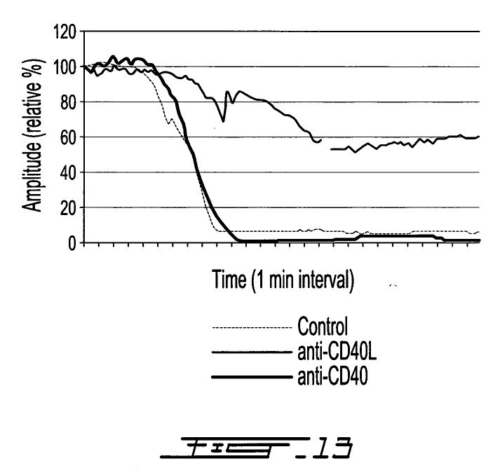

Thrombosis is the formation of a thrombus inside a blood vessel, obstructing blood flow. A thrombus is physiologic in cases of injury, but pathologic in case of thrombosis. Inflammation shifts haemostatic mechanisms in favor of thrombosis. CD40L, for example via its interaction with αllbβ3 on platelets, was found to be implicated in the formation of a stable thrombus (82). As described in Example 1, a system has been developed to study the role CD40L interfering agents' by studying their impact on thrombosis. This system has proved efficient in detecting the effect of CD40L interfering agents or molecules. Indeed, when thrombosis was studied in the presence of anti-CD40 mAbs and anti-CD40L mAbs (2 mice were used for each group) results showed that while treatment with anti-CD40 did not prevent the development of thrombosis, treatment with anti-CD40L completely abolished thrombosis (FIG. 13).

### Example 11

### sCD40L Y169/G226 Monomers Prevent Thrombosis in Mice

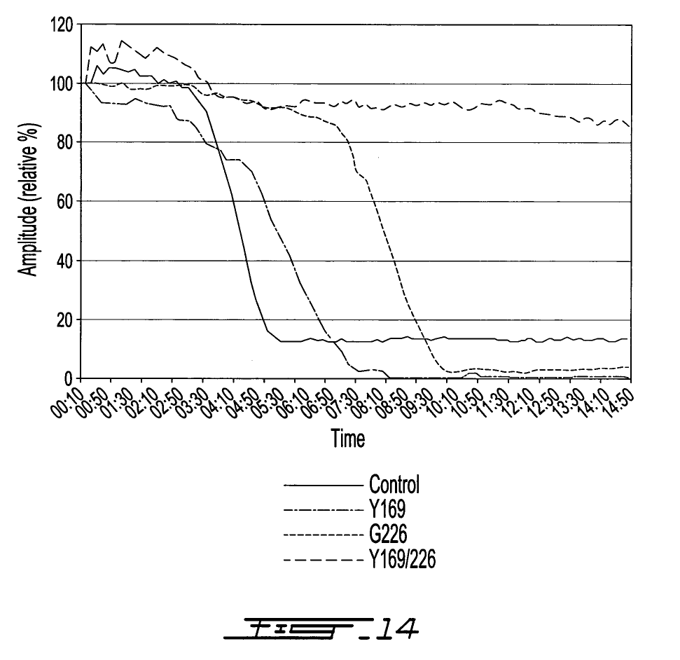

The usefulness of monomeric CD40L mutants that are still able to bind to their receptors without triggering any intracellular signal (described in Example 1 and 9), as inhibitors of CD40L interactions for the prevention of thrombosis was investigated. In this experiment, monomeric soluble murine CD40L (described in Example 1) were used. The results are shown in FIG. 14. Thrombosis was impacted by treatment with smCD40L Y169 SEQ ID NO.10 monomeric form or G226 SEQ ID NO.11 monomeric form as shown by the amplitude (%) of blood flow plotted against time. Interestingly, substitution of both residues (Y169 and G226 SEQ ID NO.12) with alanines completely inhibited the FeCl₃-induced thrombosis. Thus, monomeric CD40L, for example a double mutant in residues Y169 (Y170 in human) and G226 (G227 in human) represent a tool of great value in the treatment of CD40L-related various inflammatory and autoimmune disorders.

### Example 12

### CD40L Antibody Diminishes Bronchial Hyper-Responsiveness

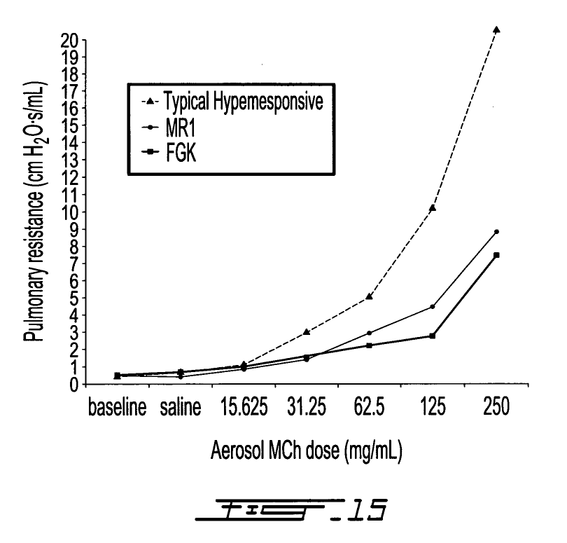

The impact of blocking CD40L in bronchial hyperresponsiveness was studied. Results are shown in FIG. 15. Mice were sensitized and challenged with OVA, and treated with 5 intranasal instillations of 20 μg anti-CD40L (MR1) or anti-CD40 (FGK) antibody. These two mAbs are known to block serveral cellular responses mediated by CD40/CD40L interaction. Pulmonary resistance was measured by flexiVent, after increasing concentrations of aerosolized methacholine (15.6-250 mg/ml). Data are compared to typical levels of hyperresponsiveness seen in sensitized and challenged animals. Treatment with anti-CD40L and anti-CD40 mAbs diminished bronchial hyperresponsiveness in treated animals compared to untreated animals, showing that both CD40 and CD40L molecules are involved in OVA-induced hyperresponsiveness. As such blocking CD40L using agent of the present invention will help in controlling bronchial hyperresponsiveness.

### Example 13

### CD40L Antibody Impacts IL-6 Expression in Asthmatic Patients

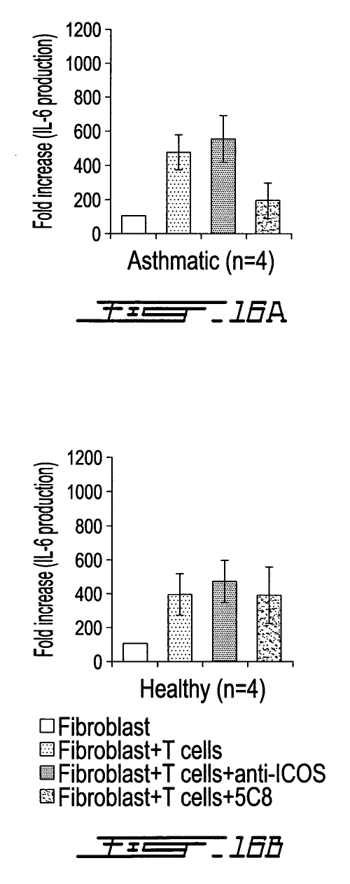

Bronchial fibroblast from asthmatic and non-asthmatic subjects were co-cultured with T cell lymphocytes in the absence or presence of anti-CD40L mAbs 5C8 or anti-ICOS mAbs and IL-6 production was measured by PCR. IL-6 expression was observed in co-cultured cells derived from asthmatic and non-asthmatic patients as shown in FIG. 16. However, treatment with anti-CD40L mAbs significantly prevented IL-6 expression from T cell stimulated bronchial fibroblast of asthmatic patients but had no effect on T-cell stimulated fibroblast of healthy donors. As such, the expression of IL-6 in asthmatic patient appeared to be mainly mediated by the interaction of CD40 ligand with its receptor(s) whereas the IL-6 expression observed in healthy donors is mediated by CD40L-independent interaction.

Although the present invention has been described hereinabove by way of specific embodiments thereof, it can be modified, without departing from the spirit and nature of the subject invention as defined in the appended claims.

### REFERENCES

1van Kooten, C., and J. Banchereau. 2000. J Leukoc Biol 67:2-17
2 Van Kooten, C., and J. Banchereau. 1996. Adv Immunol 61:1-77
3 Schonbeck, U., and P. Libby. 2001. Cell Mol Life Sci 58:4
4 Grammer, A. C., and P. E. Lipsky. 2000. Adv Immunol 76:61-178
5 Howard, L. M., and S. D. Miller. 2004. Autoimmunity 37:411
6 Toubi, E., and Y. Shoenfeld. 2004. Autoimmunity 37:457
7 Andre, P. et al., 2002. Circulation 106:896
8 Prasad, K. S. et al., 2003. Proc Natl Acad Sci USA 100:12367
9 Shattil, S. J., and P. J. Newman. 2004. Blood 104:1606
10 van der Flier, A., and A. Sonnenberg. 2001. Cell Tissue Res 305:285
11 Mould, A. P., and M. J. Humphries. 2004. Curr Opin Cell Biol 16:544
12 Kawai, T. et al., 2000. Nat Med 6:114
13 Yasuda et al., 1995. International Immunology, 7: 251-8
14 Pulai et al., 2005. J. Immunol., 174: 5781-88
15 Zeisel et al, 2005. Arthritis Res Ther, 7(1): R118-126
16 Harris, E. D., New England Journal of Medicine, Vol. 322, p. 1277-1289 (1990)
17 Cash, J. M., et al., NEJM, Vol. 330, p. 1368-1375 (1994)
18 Burrage et al., 2006. Front Biosci., 11: 529-543
19 Milner and Cawston, 2005. Curr. Drug Targets lnflamm Allergy, 4(3) 363-75
20 Spriggs et. al., JEM 176,1543-1550 (1992)
21 Wei et al., Endocrinology 142,1290-1295, (2001)
22 Srivastava et al., JBC 276,8836-8840 (2001)
23 Gauld et. al., J. Immunol. 168, 3855-3864 (2002)
24 Acsadi et al. (1991) Nature 332:815-818;
25 Wolff et al. (1990) Science 247:1465-1468
26 Wu, G. and Wu, C. H. (1988) J. Biol. Chem. 263:14621
27 Wilson el al. (1992) J. Biol. Chem. 267:963-967
28 U.S. Pat. No. 5,166,320
29 Curiel el al. (1991) Proc. Natl. Acad. Sci. USA 88:8850;
30 Cristiano et al. (1993) Proc. Natl. Acad. Sci. USA 90:2122-2126
31 Miller, A. D. (1990) Blood 76:271
32 Current Protocols in Molecular Biology, Ausubel, F. M. et al. (eds.) Greene Publishing Associates, (1989), Sections 9.10-9.14
33 Eglitis, et al. (1985) Science 230:1395-1398
34 Danos and Mulligan (1988) Proc. Natl. Acad. Sci. USA 85:6460-6464
35 Wilson et al. (1988) Proc. Natl. Acad. Sci. USA 85:3014-3018
36 Armentano et al. (1990) Proc. Natl. Acad. Sci. USA 87:6141-6145
37 Huber et al. (1991) Proc. Natl. Acad. Sci. USA 88:8039-8043
38 Ferry et al. (1991) Proc. Natl. Acad. Sci. USA 88:8377-8381
39 Chowdhury et al. (1991) Science 254:1802-1805;
40 van Beusechem et al. (1992) Proc. Natl. Acad. Sci. USA 89:7640-7644
41 Dai et al. (1992) Proc. Natl. Acad. Sci. USA 89:10892-10895
42 Hwu et al. (1993) J. Immunol. 150:4104-4115
43 U.S. Pat. No. 4,868,116; U.S. Pat. No. 4,980,286
44 PCT Application WO 89/07136
45 PCT Application WO 89/02468
46 PCT Application WO 89/05345
47 PCT Application WO 92/07573
48 Berkner et al. (1988) BioTechniques 6:616
49 Rosenfeld et al. (1991) Science 252:431-434
50 Rosenfeld et al. (1992) Cell 68:143-155
51 Lemarchand et al. (1992) Proc. Natl. Acad. Sci. USA 89:6482-6486
52 Herz and Gerard (1993) Proc. Natl. Acad. Sci. USA 90:2812-2816
53 Quantin el al. (1992) Proc. Natl. Acad. Sci. USA 89:2581-2584
54 Muzyczka et al. Curr. Topics in Micro. and Immunol. (1992) 158:97-129
55 Flotte et al. (1992) Am. J. Respir. Cell. Mol. Biol. 7:349-356
56 Samulski et al. (1989) J. Virol. 63:3822-3828;
57 McLaughlin et al. (1989) J. Virol. 62:1963-1973
58 Tratschin et al. (1985) Mol. Cell. Biol. 5:3251-3260
59 Hermonat et al. (1984) Proc. Natl. Acad. Sci. USA 81:6466-6470
60 Wondisford et al. (1988) Mol. Endocrinol. 2:32-39
61 Tratschin et al. (1984) J. Virol. 51:611-619
62 Flotte et al. (1993) J. Biol. Chem. 268:3781-3790
63 U.S. Pat. No. 5,399,346
64 PCT Publication WO 95/05452
65 Clapp, D. W., et al., Blood 78: 1132-1139 (1991)
66 Anderson, Science 288:627-9 (2000)
67 Cavazzana-Calvo et al., Science 288:669-72 (2000)
68 Clements et al, (1975) Int J Cancer 16(1):125-33
69 Morris, A. E. et al. (1999) J Biol Chem 274, 418-423
70 Pickering J. G. et al. (2000) Am. J. Pathol. 156: 453-465
71 Wilkins, J. A., et al. (1996) J Biol Chem 271, 3046-3051
72 Stupack, D. G., and Cheresh, D. A. (2002) J Cell Sci 115, 3729-3738
73 Krokhin, O. V., et al. (2003) Biochemistry 42, 12950-12959
74 Leveille, C. et al. (1999) Eur J Immunol 29, 3516-3526
75 Ni, H. et al. (1998) J Biol Chem 273, 7981-7987
76 Lishko, V. K. et al. (2003) Exp Cell Res 283, 116-126
77 Hynes, R. O. (2002) Cell 110, 673-687
78 Hostager, B. S. et al. (2000) J Biol Chem 275, 15392-15398
79 Reyes-Moreno, C. et al. (2004) J Biol Chem 279, 7799-7806
80 Kay et al. (1992) Human Gene Therapy 3:641-647 (entre 40 et 41)
81 Pietravalle, F et al. J Biol Chem 1996. 271: 5965-5967
82 Andre, P et al. Nat Med 2002. 8: 247-252
83 Karpusas et al. Structure October 1995 3: 1031-1039
84 Ho, S N et al. (1989) Gene (Amst.) 77, 51-59

1-10. (canceled)

11. A composition for blocking the interaction between CD40L and α5β1 integrin comprising a protein, an antibody or antibody fragment capable of blocking the interaction between CD40L and α5β1 integrin and a pharmaceutically acceptable carrier.

12. A method for blocking the interaction between CD40L and α5β1 integrin comprising the step of administering an effective amount of a protein, an antibody or antibody fragment capable of blocking the interaction between CD40L and α5β1 integrin.

13-19. (canceled)

20. A method for identifying a compound capable of blocking the interaction between CD40L and α5β1 integrin, said method comprising a) measuring the binding of CD40L to α5β1 integrin in the presence versus the absence of said agent, wherein a lower binding of CD40L to α5β1 integrin in the presence of said agent is indicative that said agent is capable of blocking the interaction between CD40L and α5β1 integrin or b) measuring a CD40L-mediated α5β1 integrin activity in the presence or absence of said agent, wherein a lower α5β1 integrin activity in the presence of said agent is indicative that said agent is blocking CD40L the interaction between CD40L and α5β1 integrin.

21. (canceled)

22. The method of claim 20, wherein said CD40L-mediated α5β1 integrin activity is selected from the group consisting of production of an inflammatory mediator and cytoskeleton recruitment.

23. The method of claim 22 wherein the inflammatory mediator is selected from the group consisting of IL-6, IL-8 and metalloproteinase.

24. The method of claim 23, wherein said metalloproteinase is selected from MMP-1, MMP-2 and MMP-9.

25. A monomeric soluble form of CD40L selected from the group consisting of hCD40L mutated in its Y170 residue SEQ ID NO.4, hCD40L mutated in its G227 residue SEQ ID NO.5, hCD40L mutated in both its Y170 and G227 residue SEQ ID NO.6, mCD40L mutated in its Y169 residue SEQ ID NO.10, mCD40L mutated in its G226 residue SEQ ID NO.11, mCD40L mutated in both its Y169 and G226 residue SEQ ID NO. 12 and portion thereof.

26. The monomeric soluble form of CD40L of claim 25 wherein said mutation is replacement of the residue to an alanine residue.

27-28. (canceled)

29. The composition of claim 11, wherein the antibody is selected from the group consisting of a monoclonal antibody, a polyclonal antibody and fragment thereof.

30. The composition of claim 29, wherein the antibody is selected from the group of an antibody binding specifically to CD40L, an antibody binding specifically to α5β1 integrin and portion thereof.

31. The composition of claim 11 wherein the protein is a soluble protein.

32. The composition of claim 31, wherein the soluble protein is selected from the group of soluble CD40L, soluble α5β1 integrin and portion thereof.

33. The composition of claim 32, wherein soluble CD40L is selected from the group consisting of monomeric CD40L, dimeric CD40L, trimeric CD40L and portion thereof.

34. The composition of claim 33, wherein soluble CD40L is monomeric soluble CD40L.

35. The composition of claim 34, wherein the monomeric soluble CD40L is selected from the group consisting of hCD40L mutated in its Y170 residue SEQ ID NO.4, hCD40L mutated in its G227 residue SEQ ID NO.5, hCD40L mutated in both its Y170 and G227 residue SEQ ID NO.6, mCD40L mutated in its Y169 residue SEQ ID NO.10, mCD40L mutated in its G226 residue SEQ ID NO.11, mCD40L mutated in both its Y169 and G226 residue SEQ ID NO.12 and portion thereof.

36. The composition of claim 35, wherein said mutation is replacement of the residue to an alanine residue.
